(19) United States
(12) Patent Application Publication
(10) Pub. No.: US 2020/0230068 A1
(43) Pub. Date: Jul. 23, 2020

(54) COMPOSITIONS AND METHODS OF DELIVERY OF PHARMACOLOGICAL AGENTS

(71) Applicants: Y. Joseph MO, Princeton, NJ (US); Xudong YUAN, Morganville, NJ (US); NAL PHARMACEUTICAL GROUP LIMITED, CAUSEWAY BAY (HK)

(72) Inventors: Y. Joseph MO, Princeton, NJ (US); Xudong YUAN, Morganville, NJ (US)

(73) Assignee: NAL PHARMACEUTICAL GROUP LIMITED, CAUSEWAY BAY (HK)

(21) Appl. No.: 16/641,522

(22) PCT Filed: Aug. 8, 2018
(86) PCT No.: PCT/US2018/045710

§ 371 Date: Feb. 24, 2020

Related U.S. Application Data
(60) Provisional application No. 62/550,535, filed on Aug. 25, 2017.

(51) Int. Cl. A61K 9/50; A61K 45/06; A61K 31/337

(52) U.S. Cl. CPC A61K 9/5052; A61K 31/337; A61K 45/06

(57) ABSTRACT

Nanoparticles and microspheres are provided for delivering an anticancer agent or other active agents to a subject. The nanoparticles and the microspheres are formed from a core that is encased by a coating or shell that includes a somatostatin-albumin fusion protein or analogue thereof. The somatostatin-albumin fusion protein includes at least one albumin (or an analog thereof) moiety, at least one somatostatin moiety (e.g. SST-14, SST-28), and at least one spacer connecting albumin to albumin, somatostatin to somatostatin and/or albumin to somatostatin moieties.

### CROSS-REFERENCE TO RELATED APPLICATION

This application claims the benefit of U.S. Provisional Application No. 62/550,535, filed Aug. 25, 2017, the contents of which are incorporated herein by reference.

### FIELD OF THE INVENTION

This invention relates to a composition comprising a pharmacologically active ingredient encapsulated in the form of particles. In particular, the pharmaceutically active ingredient is encapsulated in a biocompatible polymeric shell that includes a recombinant fusion protein that includes an albumin domain and a somatostatin domain.

### BACKGROUND

The therapeutic efficacy of most anticancer agents is predicated on achieving adequate local delivery to the tumor site. Many cancer chemotherapeutic agents have been shown to be highly effective in vitro, but not as effective in vivo. This disparity is believed to be attributable, in part, to the difficulty in delivering drug to the tumor site at therapeutic levels and the need for high percentages of tumor cell clearance to provide an effective treatment (Jain, 1994, Scientific American 271(1):58-65; Tannock, 1998, Lancet. 351 Suppl 2:SII9-16). Therapeutic molecules, cytokines, antibodies, and viral vectors are often limited in their ability to affect the tumor because of difficulty crossing the vascular wall (Yuan, 1998, Seminars in Radiation Oncology 8(3): 164-175). Inadequate specific delivery can lead to the low therapeutic index frequently observed with current cancer chemotherapeutics.

Somatostatin (“SST”) is a polypeptide hormone secreted by a variety of endocrine and non-endocrine tissues and is widely distributed throughout the body. Somatostatin inhibits pituitary, pancreatic, and gastrointestinal hormone secretion release, as well as cytokine production, intestinal motility and absorption, vascular contractility, and cell proliferation. Recent studies have found that SST is useful as a treatment for certain cancers of the endocrine system, inhibiting tumor growth, inhibiting the proliferation of endocrine tumors, and many other solid tumors, such as breast cancer, colorectal cancer, liver cancer, lung cancer, endocrine cancer, neuroendocrine cancers, pancreatic cancer and prostate cancer. In addition, as reported by Wangberg, 1997, The Oncologist 2:50-58, SST will selectively bind to certain tumors, including neuroendrocrine tumors, that express SST receptors to which SST and therapeutic analogs of SST will preferentially bind.

The somatostatin molecule has two biologically active forms: somatostatin-14 (SST-14), the cyclic tetradecapeptide, and somatostatin-28 (SST-28), an N-terminally elongated form of SST-14. SST-14 is a cyclic peptide with a length of 14 residues, containing a disulfide linkage between cysteines at positions 3 and 14. SST-28 is an N-terminal extension form (28 residues) of the same precursor that is proteolytically cleaved to generate SST-14. Although the two forms of somatostatin have similar activity, their respective potency and histological characteristics vary. For example, SST-14 displays more pronounced inhibition of glucagon and gastrin, while SST-28 displays more pronounced inhibition of growth hormone and insulin action. Both forms of somatostatin exert their respective biological functions through SST receptors on target cells and via intracellular pathways. Five subtypes of somatostatin receptors (SSTR 1-5) have been recognized, with two spliced variants for SSTR2: SSTR2A and SSTR2B, with a different carboxyl terminus.

The beneficial effects of somatostatin in the treatment of certain hypersecretory endocrine disorders, and its anti-proliferation effect on tumors are well recognized. However, the half-life of somatostatin in vivo is only 2-3 minutes due to enzymatic degradation and endocytosis, limiting clinical utility of somatostatin. In the past decade, numerous stable somatostatin analogs have been developed. For example, octreotide and lanreotide are used in treatment of growth hormone (GH)-secreting adenomas and carcinoids.

U.S. Pat. No. 5,439,686, described substantially water insoluble ingredients, such as paclitaxel (Taxol®), formulated within particles with an outer shell comprising a biocompatible polymer, e.g., a protein such as albumin, and the particles are suspended in a biocompatible liquid.

Co-owned international patent application No. PCT/US2016/019950, with an international filing date of Feb. 26, 2016, and co-owned U.S. patent application Ser. No. 15/249,346, filed on Aug. 26, 2016, describe stable recombinant fusion proteins containing an albumin moiety and a somatostatin moiety, wherein these moieties are connected via a spacer. The fusion protein has the benefit of providing a stable somatostatin analog for treating or downregulating tumors responsive to somatostatin. However, despite this development, there remains a longstanding need in the art for compositions combining the benefits of somatostatin activity with other therapeutic or diagnostic moieties, including other types of anticancer agents.

### SUMMARY OF THE INVENTION

Accordingly, the invention provides for particles comprising a pharmacologically active ingredient, or a diagnostic ingredient, and a polymeric shell, wherein the polymeric shell comprises a somatostatin-albumin fusion protein.

In certain embodiments of the invention, the polymeric shell substantially contains the pharmacologically active agent.

In other embodiments of the invention, the polymeric shell includes from about 5 percent to about 100 percent, by weight, of somatostatin-albumin fusion protein (“SST fusion protein”), or alternatively, the polymeric shell includes from about 65 percent to about 95 percent, by weight, of a somatostatin-albumin protein. In certain aspects, the weight ratio of the SST fusion protein and the pharmacologically active ingredient, or the diagnostic ingredient, in the particles is about 20:1 to 1:20.

In another embodiment of the invention, the particle further comprises a pharmacologically active ingredient that is an anticancer agent. For example, the anticancer agent is selected from the group consisting of nitrogen mustard, nitrosourea, ethyleneimine, alkane sulfonates, tetrazine, platinum compounds, pyrimidine analogs, purine analogs, antimetabolites, folate analogs, anthracyclines, taxane, vinca alkaloid, topoisomerase inhibitor, hormonal agent, and combinations thereof. When the anticancer agent is a taxane, for example, the taxane is optionally selected from the group consisting of paclitaxel, docetaxel, camptothecin, cabazitaxel, taxinine, cephalomannine, and analogs and derivatives thereof.

Preferably, the somatostatin-albumin fusion protein of the invention comprises:

an SST;

an L; and

an ALB, that are operably connected,

wherein
L connects SST and ALB, in any order,
SST is a somatostatin, its analogue or derivative;
L is a spacer or a linker; and
ALB is an albumin, its analogue or variant.

In particular embodiments, the fusion protein is selected from the group consisting of:

SST-(L)_{X1}-ALB (I);

ALB-(L)ₓ₁-SST (II);

[SST-(L)ₓ₁]_{y1}-ALB (III);

ALB-[(L)ₓ₁-SST]_{y1} (IV);

[SST-(L)ₓ₁]_{y1}-ALB-[(L)ₓ₂-SST]_{y2} (V);

[SST-(L)ₓ₁]_{y1}-ALB-[(L)ₓ₂-SST]_{y2}-(L)ₓ₃-ALB (VI);

[SST-(L)ₓ₁]_{y1}-ALB-[(L)ₓ₂-SST]_{y2}-(L)ₓ₃-ALB-[(L)ₓ₄-SST]_{y3} (VII);

ALB-(L)ₓ₁-[SST-(L)ₓ₂]_{y1}-ALB (VIII);

ALB-(L)ₓ₁-[SST-(L)ₓ₂]_{y1}-ALB-[(L)ₓ₃-SST]_{y2}-(L)ₓ₁-ALB (IX); and

ALB-(L)ₓ₁-[S ST-(L)ₓ₂]_{y1}-ALB-[(L)ₓ₃-SST]_{y2}-(L)ₓ₁-ALB-[(L)ₓ₄-SST]_{y3} (X);

wherein, x1, x2, x3, x4, y1, y2, or y3 is independently zero or an integer selected from 1-10.

The inventive particles include a fusion protein wherein the SST is either naturally occurring or synthetically manufactured. In a particular embodiment of the invention, the SST of the fusion protein comprises one or more tandem repeats of a sequence encoding SST-14 or SST-28, represented by SEQ ID NOS: 17 or 18, respectively, or a sequence having at least 85% identity to either of these sequences. For example, the SST of the fusion protein is SST-14 or SST-28.

Further, the fusion protein includes a linker L, that is either a flexible or an alpha helically structured polypeptide linker or spacer. In a particular embodiment, L is a peptide having 2-100 amino acids. In a further embodiment, L is a peptide that contains at least one GGGGS, A(EAAAK)4A, (AP)n domain, (G)8, or (G)5, or any combination thereof, wherein n is an integer selected from 10-34.

The fusion protein also includes mammalian serum albumen (ALB). In particular embodiments, ALB is mammalian serum albumin, including, for example, ALB according to SEQ ID NO: 25, or a protein sequence having at least 85% sequence identity thereto.

In a particular embodiment of the invention, x1, x2, x3, x4 are each independently an integer selected from 1-5, and/or y1, y2, y3 are each independently an integer selected from 1-5.

In a further embodiment, the somatostatin-albumin fusion protein is substantially crosslinked by way of disulfide bonds, for example, the disulfide bonds are formed by sonication.

The inventive particles are optionally prepared so that the largest cross-sectional dimension of the polymeric shell is about 1 micron to 0.01 micron. Alternatively, the inventive particles are optionally prepared so that the largest cross-sectional dimension of the polymeric shell is from 0.4 micron to 0.01 micron.

In certain embodiments, the polymeric shell containing the pharmacologically active agent therein is suspended in a biocompatible aqueous liquid or in a biocompatible dispersing agent.

In particular, the biocompatible dispersing agent is selected from soybean oil, coconut oil, olive oil, safflower oil, cotton seed oil, aliphatic, cycloaliphatic, or aromatic hydrocarbons having 4-30 carbon atoms, aliphatic or aromatic alcohols having 2-30 carbon atoms, aliphatic or aromatic esters having 2-30 carbon atoms, alkyl, aryl, or cyclic ethers having 2-30 carbon atoms, alkyl or aryl halides having 1-30 carbon atoms, optionally having more than one halogen substituent, ketones having 3-30 carbon atoms, polyalkylene glycol, or combinations of any two or more thereof.

In another embodiment of the invention, the particle further comprises a diagnostic ingredient. The diagnostic agent is optionally selected from the group consisting of ultrasound contrast agents, radiocontrast agents, magnetic resonance image contrast agents, and combinations thereof.

In a still further embodiment, the invention also provides for a method for the delivery of substantially water insoluble pharmaceutical agents to a subject in need thereof, the method comprising administering to said subject in need thereof an effective amount of the inventive particles.

In a still further embodiment, the invention also provides for a method for preparing particles comprising pharmaceutically active ingredients, comprising:

subjecting an aqueous medium containing a somatostatin-albumin fusion protein and a pharmaceutically active agent to shear conditions for a time sufficient to promote crosslinking of the somatostatin-albumin fusion protein by disulfide bonds to produce a polymeric shell containing the pharmacologically active agent therein. The pharmaceutically active agent is optionally dispersed in a dispersing agent. The shear conditions are provided, for example, by homogenizing the aqueous medium containing a somatostatin-albumin fusion protein and a pharmaceutically active agent under static mixing, high pressure homogenization, microfluidization conditions in the range of about 10 up to 100,000 psi.

The pharmaceutically active ingredient is optionally an anticancer agent that is water soluble or water insoluble, that is selected from the group consisting of nitrogen mustard, nitrosoruea, ethyleneimine, alkane sulfonates, tetrazine, platinum compounds, pyrimidine analogs, purine analogs, antimetabolites, folate analogs, anthracyclines, taxane, vinca alkaloid, topoisomerase inhibitor, hormonal agent, and combinations thereof. In particular, the anticancer agent is, for example, paclitaxel or docetaxel.

In a further embodiment, when preparing the inventive particles, the shear conditions are provided, for example, by high intensity ultrasound comprising an acoustic power in the range of about 50 up to 200 watts/cm² for a time period from about 2 seconds through 5 minutes.

In preferred aspects of the invention, the inventive particles selectively bind to tumor cells via tumor somatostatin receptors.

In order to more fully appreciate the invention, the following terms are defined below.

The invention broadly provides particles of a small size, i.e., “microspheres” and/or “nanoparticles” for drug delivery. Microspheres and nanoparticles are defined based on the mean cross-sectional diameter of the included particles.

As used herein, the term “micron” refers to a unit of measure of one-thousandth of a millimeter (1 m) or 1000 nm.

“Microspheres” according to the invention are inventive particles with a mean cross-sectional diameter ranging from about 1 μm to about 1000 μm, that include a polymeric shell covering, and in whole or in part, a core that includes one or more active agents.

“Nanoparticles” according to the invention are broadly defined herein as particles with a mean cross-sectional diameter ranging from about 0.001 μm to about 1 μm.

Microspheres are larger than nanoparticles, and have the general advantage of delivering more active agent per particle and the potential to provide a prolonged or controlled release of an active agent, and can be readily administered by injection into tissues, e.g., as a subdermal or intramuscular injection. However, microspheres have certain disadvantages for intravenous administration, e.g., a tendency to aggregate or form clumps after injection, and for larger microspheres, potential difficulties in circulating through capillary beds. Nanoparticles, particularly those smaller than 0.4 μm, have advantages relative to microspheres, particularly for intravenous injection, e.g., nanoparticles are less likely to aggregate, and are more likely to avoid the reticuloendothelial system (RES), are able to enter cells via pinocytosis, and have an advantage of targeting and accumulating in tumor tissues based on the enhanced permeability and retention (EPR) effect in solid tumors. The EPR effect is in addition to the selective binding and targeting of the SST fusion protein component of the polymer shell to those tumors that present SST receptors.

It should also be understood that singular forms such as “a,” “an,” and “the” are used throughout this application for convenience, however, except where context or an explicit statement indicates otherwise, the singular forms are intended to include the plural.

All numerical ranges should be understood to include each and every numerical point within the numerical range, and should be interpreted as reciting each and every numerical point individually. The endpoints of all ranges directed to the same component or property are inclusive, and intended to be independently combinable.

As used herein, the term “about” means within 10% of the reported numerical value, and preferably within 5% of the reported numerical value.

The phrase “consisting essentially of” means that the composition or method may include additional ingredients and/or steps, but only if the additional ingredients and/or steps do not materially alter the basic and novel characteristics of the claimed composition or method, i.e., the additional ingredient(s) and/or step(s) would serve no purpose material to the claimed composition or method.

As used herein, the term “biocompatible” describes a substance that does not appreciably alter or affect in any adverse way, the biological system into which it is introduced.

As used herein, the terms, “co-administered” or “co-administration” with the inventive particles, and one or more other active agents, are intended to encompass the administration of such other active agent, together the inventive particles, to a subject, whether administered simultaneously with the particles, or before or after the administration of the particles. Broadly, the inventive particles deliver one or more active agents, and such active agent or agents may provide a coordinated and/or synergistic effect when administered to a subject who is also being administered one or more other active agents that are not contained in the inventive particles.

As used herein, the term “subject” is meant to refer to any animal to which the inventive particles are administered, and preferably the animal is a mammal. An animal subject can include a human subject, or a non-human subject. Without limitation, a non-human, or animal subject is any animal to which the inventive particles may be administered, e.g., during the course of the care or treatment of either a domestic or wild animal. Non-human subjects preferably include domesticated mammals, such as members of the genus Canis (dogs, wolves, coyotes, and jackals), members of the genus Felis (e.g., the domestic cat), members of the genus Camelus (camels), members of the genus Equus (e.g., horses, asses, and zebras), members of the subfamily Caprinae (sheep and goats) and/or members of the subfamily Bovinae (ungulates such as domestic cattle, bison, African buffalo, water buffalo, yak, and the four-horned and spiral-horned antelopes). A non-human subject is also contemplated to include, for example, a domesticated avian, such as farmed fowl, e.g., chickens, ducks, turkeys, geese, ostrich and the like, and/or pet avians, such as finches and/or members of the order of Psittaciformes, e.g., parrots and parakeets.

Before the present invention is described in detail below, it is to be understood that this invention is not limited to the particular methodology, protocols and reagents described herein, as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art.

### BRIEF DESCRIPTION OF THE DRAWING

FIG. 1 illustrates the release profiles of paclitaxel from Somatostatin (SST)-Human Serum Albumin(HSA) paclitaxel particles and Abraxane particles in vitro. Time in hours is along the X-axis and percent release is along with Y-axis. The curve labeled with diamonds (♦) marks the release of Paclitaxel from SST-HSA particles. The curve labeled with squares (▪) marks the release of Abraxane.

### DETAILED DESCRIPTION OF THE INVENTION

The invention provides particles containing at least one active agent, and a polymeric shell that includes surrounds and encapsulates the active agent. Optionally, a portion of the active agent is exposed to the medium or is outside the polymeric shell. The polymeric shell includes, in whole or in part, a somatostatin-albumin fusion protein. In one embodiment, the somatostatin-albumin fusion protein is substantially crosslinked by disulfide bonds. The particles are also optionally suspended in a pharmaceutically compatible carrier, such as a physiologically acceptable buffered solution.

### Active Agents

The inventive particles include one or more pharmacologically active ingredients, that may also be referred to herein, without limitation, as active agents, as therapeutic agents or as active pharmaceutical ingredient(s) (APIs). The active agent may be a physiologically or pharmacologically active substance that can produce a desired biological effect in a subject, such as an animal subject, including a human. The term “active agent” is also intended to encompass a diagnostic agent, or an active agent of nutritional value. The selection of a particular agent depends on the desired application. The term “active agent” is also intended to encompass a precursor to an active agent that is converted to the active form in vivo, e.g., a prodrug or other precursor. The active agent may be an inorganic or organic compound, including peptides, proteins, nucleic acids, and small molecules. The active agent may be in various forms, such as an unchanged molecule, molecular complex, pharmacologically acceptable salt, such as hydrochloride, hydrobromide, sulfate, laurate, palmitate, phosphate, nitrite, nitrate, borate, acetate, maleate, tartrate, oleate, salicylate, and the like. For an acidic therapeutic agent, salts of metals, amines or organic cations, for example, quaternary ammonium, may be used. Derivatives of drugs, such as bases, esters and amides also may be used as an active agent. An active agent that is water insoluble may be used in a form that is a water soluble derivative thereof, or as a base derivative thereof, which in either instance, or by its delivery, is converted by enzymes, hydrolyzed by the body pH, or by other metabolic processes to the original therapeutically active form.

The active agent may be an anticancer agent, such as a chemotherapeutic agent. The active agent may also be an immunosuppressive agent, a cytokine, a cytotoxic agent, a nucleolytic compound, a radioactive isotope, a receptor, an anti-inflammatory agent, an analgesic agent, an antibiotic agent, an antiviral agent, an antifungal agent, an antiparasitic agent, and/or any combination thereof.

Examples of pharmaceutically active anticancer agents, for inclusion in the inventive particles, and/or for co-administration with the inventive particles, are listed by U.S. Pat. No. 8,173,115, incorporated by reference herein, and include, broadly, nitrogen mustards, nitrosoureas, ethyleneimine, alkane sulfonates, tetrazine, platinum compounds, pyrimidine analogs, purine analogs, antimetabolites, folate analogs, anthracyclines, taxanes, vinca alkaloids, topoisomerase inhibitors and hormonal agents.

Particular exemplary chemotherapy drugs include, for example, actinomycin-d, alkeran, Ara-C(arabinosylcytosine), anastrozole, asparaginase, bicnu, bicalutamide, bleomycin, busulfan, capecitabine, carboplatin, carboplatinum, carmustine, CCNU, chlorambucil, cisplatin, cladribine, CPT-11 (irinotecan), cyclophosphamide, cytarabine, cytosine arabinoside, cytoxan, dacarbazine (DTIC), dactinomycin, daunorubicin, dexrazoxane, docetaxel, doxorubicin, epirubicin, ethyleneimine, etoposide, floxuridine, fludarabine, fluorouracil, flutamide, fotemustine, gemcitabine, herceptin, hexamethylamine, hydroxyurea, idarubicin, ifosfamide, lomustine, mechlorethamine, melphalan, mercaptopurine, methotrexate, mitomycin, mitotane, mitoxantrone, oxaliplatin, paclitaxel, pamidronate, pentostatin, plicamycin, procarbazine, rituximab, streptozocin, imatinib (STI-571, Gleevec®, Glivec®), streptozocin, tamoxifen, temozolomide, teniposide, tetrazine, thioguanine, thiotepa, tomudex, topotecan, treosulphan, trimetrexate, vinblastine, vincristine, vindesine, vinorelbine, VP-16, and xeloda.

Useful anticancer drugs contemplated to be included in the inventive particles, or co-administered with the inventive particles, also include alkylating agents, such as thiotepa and cyclosphosphamide; alkyl sulfonates, such as busulfan, improsulfan and piposulfan; aziridines, such as benzodopa, carboquone, meturedopa, and uredopa; ethylenimines and methylamelamines, including altretamine, triethylenemelamine, trietylenephosphoramide, triethylenethiophosphaoramide and trimethylolomelamine; nitrogen mustards, such as chlorambucil, chlornaphazine, cholophosphamide, estramustine, ifosfamide, mechlorethamine, mechlorethamine oxide hydrochloride, melphalan, novembiehin, phenesterine, prednimustine, trofosfamide, uracil mustard; nitroureas, such as cannustine, chlorozotocin, fotemustine, lomustine, nimustine, and ranimustine; antibiotics, such as aclacinomysins, actinomycin, authramycin, azaserine, bleomycins, cactinomycin, calicheamicin, carabicin, carminomycin, carzinophilin, chromoinycins, dactinomycin, daunorubicin, detorubicin, 6-diazo-5-oxo-1-norleucine, doxorubicin, epirubicin, esorubicin, idambicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites such as methotrexate and 5-fluorouracil (5-FU); folic acid analogues, such as denopterin, methotrexate, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, floxuridine, and 5-FU; androgens, such as calusterone, dromostanolone propionate, epitiostanol, rnepitiostane, and testolactone; anti-adrenals, such as aminoglutethimide, mitotane, and trilostane; folic acid replenisher, such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK™; razoxane; sizofrran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotEPa; taxoids, e.g., paclitaxel (Taxol®, Bristol-Myers Squibb Oncology, Princeton, N.J.), doxetaxel (Taxotere®, Rhone-Poulenc Rorer, Antony, France), and cabazitaxel(Jevtana®, Sanofi-Aventis); chlorambucil; gemcitabine; 6-thioguanine; mercaptopurine; methotrexate; platinum analogs, such as cisplatin and carboplatin; vinblastine; platinum; etoposide (vp-16); ifosfamide; mitomycin c; mitoxantrone; vincristine; vinorelbine; navelbine; novantrone; teniposide; daunomycin; aminopterin; xeloda; ibandronate; cpt-11; topoisomerase inhibitor RFS 2000; difluoromethylornithine (dmfo); retinoic acid; esperamicins; capecitabine; and pharmaceutically acceptable salts, acids or derivatives of any of the above. Also included are anti-hormonal agents that act to regulate or inhibit hormone action on tumors, such as anti-estrogens including for example tamoxifen, raloxifene, aromatase inhibiting 4(5)-imidazoles, 4 hydroxytamoxifen, trioxifene, keoxifene, onapristone, and toremifene (fareston); and anti-androgens such as flutamide, nilutamide, bicalutamide, leuprolide, and goserelin; and pharmaceutically acceptable salts, acids or derivatives of any of the above.

Cytokines may be also be included in the inventive particles as a therapeutic agent(s), or co-administered with the inventive particles, e.g., for co-treating or augmenting another therapeutic agent. Examples of such cytokines are lymphokines, monokines, and traditional polypeptide hormones.

Polynucleotides can be encapsulated in the inventive particles as a therapeutic agent(s). Polynuleotides include, not limited to, small or short interfering RNA (“siRNA”), micro RNA, RNA, DNA, antisense, or genes.

Traditional polypeptide hormones include, for example, growth hormones, such as human growth hormone, N-methionyl human growth hormone, and bovine growth hormone; parathyroid hormone; thyroxine; insulin; proinsulin; relaxin; prorelaxin; glycoprotein hormones, such as follicle stimulating hormone (FSH), thyroid stimulating hormone (TSH), and luteinizing hormone (LH); hepatic growth factor; fibroblast growth factor; prolactin; placental lactogen; tumor necrosis factor-α and β; mullerian-inhibiting substance; mouse gonadotropin-associated peptide; inhibin; activin; vascular endothelial growth factor; integrin; thrombopoietin (TPO); nerve growth factors, such as NGF-13; platelet growth factor; transforming growth factors (TGFs), such as TGF-α and TGF-β; insulin-like growth factor-I and -II (IGF-1 and IGF-II); prostaglandin (PG); prostaglandin E1 (PGE1, alprostadil) and prostaglandin E2 (PGE2, dinoprostone); erythropoietin (EPO); osteoinductive factors; interferons such as interferon-α, -β and -γ; colony stimulating factors (CSFs), such as macrophage-CSF (M-CSF); granulocyte-macrophage-CSF (GM-CSF); and granulocyte-CSF (GCSF); interleukins (ILs), such as IL-1, IL-la, IL-2, IL-3, IL-4, IL-5, IL-6, IL-7, IL-8, IL-9, IL-11, IL-12, IL-15; a tumor necrosis factor, such as TNF-α or TNF-β; and other polypeptide factors including LIF and kit ligand (KL). The half-life of IGF-1 is very short: about 10-20 minutes. IGF-1 can be modified to make the amino acid analog IGF-1 LR3 (long) or IGF-1 DES (truncated). IGF-1 DES is ten times more potent than IGF-1. Included among the cytokines are the interferons (IFNs), e.g., IFNα, IFNβ and IFNγ interferons, and art-known recombinant variations thereof. In particular, recombinant IFN alfa 2b (Intron®A) is contemplated to be included in particles according to the invention, or co-administered with inventive particles for certain conditions. As used herein, the term “cytokine” includes proteins from natural sources or from recombinant cell culture and biologically active equivalents of the native sequence cytokines.

For example, interferon is administered as part of the treatment of certain cancers, such as gastroenteropancreatic neuroendocrine tumors (GEP-NETS) to augment the benefits of somatostatin analogs such as octreotide. A particle according to the invention that includes a cytokine, e.g., an interferon, encapsulated within an SST-albumin fusion protein shell is contemplated to provide additional benefit to enhance targeting of the therapeutic agent.

Preferably, the anticancer agents include the poorly water soluble taxanes (as used herein, the term “taxane” is intended to include taxane analogs and prodrugs, e.g. cabazitaxel (Jevtana®), paclitaxel (Taxol®), and docetaxel (Taxotere™). Other taxane-like drugs are also contemplated to be included in the inventive particles, such as camptothecin and derivatives thereof. Other preferred anticancer drugs include, for example, phenesterine, daunorubicin, doxorubicin, mitotane, visadine, halonitrosoureas, anthrocylines, ellipticine, diazepam, and the like, and pharmaceutically acceptable salts, acids or derivatives of any of the above.

Other anticancer drugs contemplated to be included in the particles of the present invention, and/or co-administered with the inventive particles, include the following.

Drugs approved for treating pancreatic cancer, such as, erlotinib hydrochloride everolimus, fluorouracil, gemcitabine hydrochloride, irinotecan hydrochloride liposome, mitomycin c, paclitaxel albumin-stabilized nanoparticle formulation, and/or sunitinib malate and pharmaceutically acceptable salts, acids or derivatives of any of the above.

Drugs approved for treating gastroenteropancreatic neuroendocrine tumors, such as, lanreotide acetate, cisplatin and/or etoposide, and pharmaceutically acceptable salts, acids or derivatives of any of the above.

Drugs approved for treating thyroid cancer, such as, cabozantinib-s-malate, doxorubicin hydrochloride, lenvatinib mesylate, sorafenib tosylate and/or vandetanib, and pharmaceutically acceptable salts, acids or derivatives of any of the above.

Drugs approved for treating pituitary cancer, such as, cabergoline and/or bromocriptine, and pharmaceutically acceptable salts, acids or derivatives of any of the above.

Anesthetics contemplated to be included in the inventive particles, and/or co-administered with the inventive particles, include agents such as methoxyfluorane, isofluorane, enfluorane, halothane, benzocaine, dantrolene, barbiturates, and the like, and pharmaceutically acceptable salts, acids or derivatives of any of the above.

Other pharmaceutical agents contemplated for inclusion in the inventive particles, and/or co-administered with the inventive particles, include, simply by way of example, non-steroid anti-inflammatory agents, such as, ibuprofen, piroxicam, acetylsalicylic acid, choline, sodium and magnesium salicylates, celecoxib, diclofenac sodium/epolamine, diflunisal, etodolac, fenoprofen calcium, flurbiprofen, indomethacin, ketoprofen, ketorolac, meclofenamate sodium, mefenamic acid, meloxicam, nabumetone, naproxen, naproxen sodium, oxaprozin, rofecoxib, salsalate, sulindac, tolmetin sodium, and/or valdecoxib, and pharmaceutically acceptable salts, acids or derivatives of any of the above.

Further pharmaceutical agents contemplated for inclusion in the inventive particles, and/or co-administered therewith, include, simply by way of example, H2 blocking antacids, such as cimetidine, famotidine, ranitidine; substantially water insoluble steroids dexamethasone, methylprednisolone, prednisone, cortisone, prednisolone, triamcinolone, diflorasone, betamethasone and/or testosterone

In addition, also contemplated for inclusion in the inventive particles, and/or co-administered therewith, are substantially water insoluble immunosuppressive agents, such as, for example, cyclosporines, azathioprine, FK506, prednisone, mycophenolic acid, lefunomide, teriflunomide, tacrolimus, cyclosporine, everolimus and/or sirolimus, rivaroxaban and the like.

Also contemplated for inclusion in the inventive particles, and/or co-administered therewith, are antimicrobials, such as antibiotic agents, antiviral agents, antifungal agents, anti-blood clotting agent, anti-thrombosis agents, and/or antiparasitic agents.

Antibiotics (antibacterial) agents include, for example any molecule which exhibits a bactericidal or bacteriostatic effect. Included within the term are, for example: classic antibiotics, e.g., amphotericin B chloramphenicol, erythromycin, lincomycin, fusidic acid, streptomycin, moxifloxicin other aminoglycoside antibiotics, tetracyclines, polymyxins, fosfomycin, vancomycin, ristocetin, bacitracin, gramacidin, penicillins, and cephalosporins; antimetabolites, e.g., sulfonamides and trimethoprim; and other bactericidal or bacteriostatic agents such as small molecule toxins, radioactive compounds, and nucleoside analogues.

Antiviral agents include, for example, idoxuridine, acyclovir, ganciclovir, amantadine, rimantadine, oseltamivir, zanamivir, nevirapine, delavirdine, efavirenz, zidovudine, didanosine, zalcitabine, stavudine, lamivudine, abacavir, emtricitabine, amprenavir, fosamprenavir, indinavir, ritonavir, saquinavir, nelfinavir, tenofovir and/or adefovir.

Antifungal agents include, for example, systemic antimycotics, such as amphotericin B, voriconazole, posaconazole and/or fluconazole, and for example, topical antimycotics such as amorolfine, butenafine, butoconazole, carbolfuchsin, ciclopirox, clioquinol, clotrimazole, econazole, fluconazole, griseofulvin, itraconazole, ketoconazole, miconazole, naftifine, nystatin, oxiconazole, sulconazole, terbinafine, terconazole, tioconazole and/or tolnaftate.

Antiparasitic agents include, for example, albendazole, amphotericin B, eflornithine, fumagillin, melarsoprol, metronidazole, miltefosine, niclosamide, nitazoxanide, tinidazole, praziquantel, rifampin.

Examples of diagnostic agents contemplated for use in the practice of the present invention include ultrasound contrast agents, radiocontrast agents (e.g., iodo-octanes, halocarbons, renografin, and the like), magnetic contrast agents (e.g., fluorocarbons, lipid soluble paramagnetic compounds, quantum dots, and the like), as well as other diagnostic agents which cannot readily be delivered without some physical and/or chemical modification to accommodate the substantially water insoluble nature thereof.

Examples of agents of nutritional value contemplated for use in the practice of the present invention include amino acids, sugars, proteins, carbohydrates, fat-soluble vitamins (e.g., vitamins A, D, E, K, and the like), fat, nutraceuticals such as curcumin, and/or combinations thereof.

The agent included in the inventive particles can be water-insoluble or substantially water-insoluble.

The inventive particles are preferably employed for in vivo delivery of a substantially water insoluble active agent. As used herein, the term “in vivo delivery” refers to delivery of a pharmacologically active agent by such routes of administration as oral, intravenous, subcutaneous, intraperitoneal, intrathecal, intramuscular, inhalational, topical, transdermal, suppository (rectal), pessary (vaginal), and the like.

The included agent can be a solid or liquid, and substantially or completely contained within the polymeric shell.

Accordingly, particles of active agents are nanoparticles that can be contained within a shell having a cross-sectional diameter ranging from about 1 micron through about 0.001 microns, or less. Nanoparticles with a cross-sectional diameter of less than 0.5 microns are more preferred, while a cross-sectional diameter of less than 0.2 microns is presently the most preferred cross-sectional diameter for nanoparticles to be administered by the intravenous route of administration. In certain embodiments, the inventive nanoparticles are preferred to range in size from about 0.05 microns to about 0.3 microns, depending on the way that the nanoparticles are prepared and the purpose for which they will be used.

In another embodiment, the somatostatin-albumin fusion protein is selectively crosslinked through the formation of disulfide bonds through, for example, the amino acid cysteine that occurs in the natural structure of the protein. For example, a sonication process is used to disperse a dispersing agent containing dissolved or suspended pharmacologically active agent into an aqueous solution of the somatostatin-albumin fusion protein bearing sulfhydryl or disulfide groups whereby a shell of crosslinked somatostatin-albumin fusion protein is formed around fine droplets of non-aqueous medium. The sonication process produces cavitation in the liquid that causes tremendous local heating and results in the formation of superoxide ions that crosslink the polymer by oxidizing the sulfhydryl residues (and/or disrupting existing disulfide bonds) to form new, crosslinking disulfide bonds.

Exemplary ranges for the somatostatin-albumin fusion protein-drug preparations are protein to drug ratios (w/w) of 0.01:1 to about 100:1. More preferably, the ratios are in the range of 0.02:1 to about 40:1. While the ratio of the fusion protein to pharmaceutical agent will have to be optimized for different protein and pharmaceutical agent combinations, generally the ratio of the fusion protein to pharmaceutical agent is about 18:1 or less (e.g., about 15:1, about 10:1, about 5:1, or about 3:1). More preferably, the ratio is about 0.2:1 to about 12:1. Most preferably, the ratio is about 1:1 to about 9:1. Preferably, the formulation is essentially free of Cremophor®, and more preferably free of Cremophor EL® (BASF). Cremophor® is a non-ionic emulsifying agent that is a polyether of castor oil and ethylene oxide.

A further embodiment provides a method for the formation of the inventive particles by a solvent evaporation technique from an oil-in-water emulsion prepared under conditions of high shear forces (e.g., sonication, high pressure homogenization, or the like). This method can be conducted without the use of any conventional surfactants, and without the use of any polymeric core material to form the matrix of the particle. Instead, a somatostatin-albumin fusion protein is employed as a stabilizing agent.

The invention also provides a method for the reproducible formation of unusually small particles, i.e., nanoparticles, with a cross sectional diameter of less than 0.2 microns, which can optionally be sterile-filtered through a 0.22 micron filter. This is achieved, for example, by addition of a water soluble solvent (e.g., ethanol) to the organic phase and by carefully selecting the type of organic phase, the phase fraction and the drug concentration in the organic phase. The ability to form nanoparticles of a size that is filterable by 0.22 micron filters is advantageous, since formulations which contain a significant amount of any protein (e.g., albumin), cannot be sterilized by conventional methods such as autoclaving, due to the heat coagulation of the protein.

The invention further provides a drug delivery system in which part of the molecules of pharmacologically active agent are bound to the somatostatin-albumin fusion protein, and are therefore immediately bioavailable upon administration to a subject. The other portion of the pharmacologically active agent is contained within particles coated by the fusion protein. The particles containing the pharmacologically active agent are present as a pure active component, without dilution by any polymeric matrix.

In accordance with the present invention, there are also provided nanoparticles of submicron mean cross-sectional diameter, in powder form, which can easily be reconstituted in water or saline. The powder is obtained after removal of water by lyophilization. The somatostatin-albumin fusion protein serves as the structural component of the inventive nanoparticles, and also as a cryoprotectant and reconstitution aid. The preparation of particles filterable through a 0.22 micron filter according to the invention method as described herein, followed by drying or lyophilization, produces a sterile solid formulation useful for intravenous injection.

While it is recognized that particles produced according to the invention can be either crystalline, amorphous, or a mixture thereof, it is generally preferred that the drug be present in the formulation in an amorphous form. This would lead to greater ease of dissolution and absorption, resulting in better bioavailability.

The somatostatin-albumin fusion proteins of the certain embodiment of the invention include variants of albumin including human serum albumin and/or derivatives of somatostatin. The spacers of another embodiment of the invention encompasses peptides covalently linked to somatostatin on one terminal and albumin on another terminal. The spacers in other embodiments of the invention include peptide sequences having 2-100 amino acids.

### SST Fusion Proteins

The somatostatin-albumin fusion proteins employed in the particles of the present invention, vectors and host cells for producing the same, as well as method of purification of the proteins, are described in detail by co-owned international patent application No. PCT/US2016/019950, with an international filing date of Feb. 26, 2016, and by co-owned U.S. patent application Ser. No. 15/249,346, filed on Aug. 26, 2016.

The somatostatin-albumin fusion proteins, and analogues thereof, are prepared to include an albumin (or an analog thereof) moiety, a somatostatin moiety (e.g., SST-14, SST-28), and a spacer separating the two moieties. Variants of albumin, including human serum albumin and/or derivatives of somatostatin are also contemplated as part of the fusion proteins. Spacers within the fusion proteins include peptide sequences ranging in size from about 2 to about 100 amino acid residues.

In one embodiment, the employed fusion protein comprises:

an SST;

an L; and

an ALB,

wherein,
SST is a somatostatin or its analogues or derivatives;
L is a spacer or a linker;
the ALB is an albumin or its analogues or variants.

In certain embodiments, the fusion protein of the present invention is selected from among formulas I-X, as follows.

SST-(L)ₓ₁-ALB (I);

ALB-(L)ₓ₁-SST (II);

[SST-(L)ₓ₁]_{y1}-ALB (III);

ALB-[(L)ₓ₁-SST]_{y1} (IV);

[SST-(L)ₓ₁]_{y1}-ALB-[(L)ₓ₂-SST]_{y2} (V);

[SST-(L)ₓ₁]_{y1}-ALB-[(L)ₓ₂-SST]_{y2}-(L)ₓ₃-ALB (VI);

[SST-(L)ₓ₁]_{y1}-ALB-[(L)ₓ₂-SST]_{y2}-(L)ₓ₃-ALB-[(L)ₓ₄-S ST]_{y3} (VII);

ALB-(L)ₓ₁-[SST-(L)ₓ₂]_{y1}-ALB (VIII);

ALB-(L)ₓ₁-[SST-(L)ₓ₂]_{y1}-ALB-[(L)ₓ₃-SST]_{y2}-(L)ₓ₁-ALB (IX); and

ALB-(L)ₓ₁-[S ST-(L)ₓ₂]_{y1}-ALB-[(L)ₓ₃-SST]_{y2}-(L)ₓ₁-ALB-[(L)ₓ₄-SST]_{y3} (X);
wherein,
each x1, x2, x3, x4, y1, y2, or y3 is independently zero or an integer selected from 1-10, provided that there is at least one L present in the fusion protein.

In yet another embodiment, the employed albumin-somatostatin fusion protein is encoded by a nucleotide sequence comprising:

an SST;

an L; and

an ALB,
wherein,

SST is a somatostatin or its analogues or derivatives;

L is a spacer or a linker;

ALB is an albumin or its analogues or variants.

In certain embodiments, the nucleotide sequence is selected to encode an albumin-somatostatin fusion protein from among,

SST-(L)ₓ₁-ALB (I);

ALB-(L)ₓ₁-SST (II);

[SST-(L)ₓ₁]_{y1}-ALB (III);

ALB-[(L)ₓ₁-SST]_{y1} (IV);

[SST-(L)ₓ₁]_{y1}-ALB-[(L)ₓ₂-SST]_{y2} (V);

[SST-(L)ₓ₁]_{y1}-ALB-[(L)ₓ₂-SST]_{y2}-(L)ₓ₃-ALB (VI);

[SST-(L)ₓ₁]_{y1}-ALB-[(L)ₓ₂-SST]_{y2}-(L)ₓ₃-ALB-[(L)ₓ₄-S ST]_{y3} (VII);

ALB-(L)ₓ₁-[SST-(L)ₓ₂]_{y1}-ALB (VIII);

ALB-(L)ₓ₁-[SST-(L)ₓ₂]_{y1}-ALB-[(L)ₓ₃-SST]_{y2}-(L)ₓ₁-ALB (IX); and

ALB-(L)ₓ₁-[S ST-(L)ₓ₂]_{y1}-ALB-[(L)ₓ₃-SST]_{y2}-(L)ₓ₁-ALB-[(L)ₓ₄-SST]_{y3} (X);
wherein,
each x1, x2, x3, x4, y1, y2, or y3 is independently zero or an integer selected from 1-10, provided that there is at least one L present in the nucleotide sequence encoding an albumin-somatostatin fusion protein.

In another embodiment of the nucleotide sequence encoding the albumin-somatostatin fusion protein, the spacer sequence consists of the sequence encoding the amino acid sequence represented by SEQ ID NO: 31 or -GGGGS-.

A nucleotide sequence is also contemplated that encodes an albumin-somatostatin fusion protein comprising:

(a) a first region comprising a nucleotide sequence containing one or more adjacent repeats of a sequence encoding a human somatostatin peptide;

(b) a second region comprising a nucleotide sequence encoding human serum albumin, or a fragment thereof,

(c) a spacer region comprising a nucleotide sequence encoding a polypeptide of 2-100 residues in length;

wherein the spacer region is present between the first region and the second region, or between the first region and another first region;

wherein one or more adjacent repeats of a sequence encoding a human somatostatin peptide encodes either SST-14 or SST-28, as represented by SEQ ID NOS:17 and 18, respectively, or a sequence having at least 85% identity to either of these two sequences; or

wherein the spacer sequence consists of the sequence encoding the amino acid sequence represented by SEQ ID NO: 31 or GGGGS or by SEQ ID NO: 30 A(EAAAK)4A; or

wherein the region (a) consists of one or more adjacent repeats of either SST-14 or of SST-28, as represented by SEQ ID NOS: 23 and 24, respectively, or a sequence having at least 85% identity to either of these two sequences.

In a yet further embodiment of the nucleotide sequence encoding the albumin-somatostatin fusion protein, the first region (a) encodes a polypeptide having at least 85% sequence identity to either SEQ ID NOS: 17 or 18, SST-14, SST-28, or a fragment thereof.

In a further embodiment of the nucleotide sequence encoding the albumin-somatostatin fusion protein, the second region (b) encodes a polypeptide having at least 85% sequence identity to SEQ ID NO: 19, albumin or a fragment thereof.

Furthermore, the fusion protein employed by the present invention is contemplated to include an albumin-somatostatin fusion protein comprising:

(a) a first region comprising a polypeptide sequence of a somatostatin peptide (which may be a human somatostatin peptide);

(b) a second region comprising a polypeptide sequence of serum albumin (which may be a human serum albumin), or a fragment thereof,

(c) a spacer region comprising a polypeptide of 2-100 residues in length.

The spacer region (c) may be present between region (a) and region (b) or between region (a) and region (a). In addition, the region (a) may comprise one or more tandem repeats of a sequence encoding SST-14 or SST-28, represented by SEQ ID NOS: 17 or 18, respectively, or sequence having 85% identity to either of these sequences.

General methods for preparing suitable expression vectors and host cells are described, for example, by U.S. Pat. No. 9,296,809, incorporated by reference herein. Recombinant expression of albumin-somatostatin fusion proteins employed by the present invention, requires construction of an expression vector containing a polynucleotide that encodes the fusion protein. Once a polynucleotide encoding an albumin-somatostatin fusion protein has been obtained, the vector for the production of the albumin-somatostatin fusion protein may be produced by recombinant DNA technology using techniques well known in the art. Thus, methods for preparing a fusion protein by expressing a polynucleotide containing an albumin-somatostatin encoding nucleotide sequence are described herein. Methods which are well known to the art can be used to construct expression vectors with the appropriate transcriptional and translational control signals. These methods include, for example, in vitro recombinant DNA techniques, synthetic techniques, and in vivo genetic recombination. Thus, replicable vectors comprising a nucleotide sequence encoding the fusion protein, operably linked to a promoter, are prepared.

The prepared expression vector is transfected into a host cell by conventional techniques, and the transfected cells are then cultured by conventional techniques to produce albumin-somatostatin fusion protein. Thus, host cells containing a polynucleotide encoding the albumin-somatostatin fusion protein, operably linked to a heterologous promoter are employed.

A variety of host-expression vector systems may be utilized to express the albumin-somatostatin fusion protein. Such host-expression systems represent vehicles by which the coding sequences of interest may be produced and subsequently purified, but also represent cells which may, when transformed or transfected with the appropriate nucleotide coding sequences, express albumin-somatostatin fusion proteins in situ. Host systems are disclosed, for example by U.S. Pat. No. 8,969,538. These include, but are not limited to, microorganisms such as bacteria (e.g., E. coli, B. subtilis) transformed with recombinant bacteriophage DNA, plasmid DNA or cosmid DNA expression vectors containing albumin-somatostatin fusion protein coding sequences; yeast (e.g., Saccharomyces, Pichia) transformed with recombinant yeast expression vectors containing albumin-somatostatin fusion protein coding sequences; insect cell systems infected with recombinant virus expression vectors (e.g., baculovirus) containing albumin-somatostatin fusion protein coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing albumin-somatostatin fusion proteins coding sequences; or mammalian cell systems (e.g., COS, CHO, BHK, HEK293 cells, 3T3 cells, murine Sp2/0 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In addition to transiently transfected mammalian cells, stably transfected cells in the format of stable pools or stable cell lines which express the albumin-somatostatin fusion proteins are also included.

In a particular embodiment of the invention the inventive particles include an isolated and purified albumin-somatostatin fusion protein having the polypeptide sequences of Table 1 (e.g., a polypeptide sequence of an albumin-somatostatin fusion protein or the plasmid construct expressing such protein).

TABLE 1
A non-exclusive list of polypeptide sequences

| SEQ ID NO: | Description |
|---|---|
| SEQ ID NO: 1 | SST14-A(EAAAK)₄A-HSA-A(EAAAK)₄A-SST14 |
| SEQ ID NO: 2 | HSA-A(EAAAK)₄A-SST14 |
| SEQ ID NO: 3 | His6-GGGGS-HSA-GGGGS-SST14-HSA |
| SEQ ID NO: 4 | His6-GGGGS-HSA-GGGGS-(SST14-GGGGS)₂- |
| | HSA |
| SEQ ID NO: 5 | HSA-GGGGS-(SST14-GGGGS)₂-HSA |
| SEQ ID NO: 6 | Linker GGGGGGGG |
| SEQ ID NO: 7 | SST14-(GGGGS)₃-HSA |
| SEQ ID NO: 8 | SST14-A(EAAAK)₄A-HSA |
| SEQ ID NO: 9 | His6-GGGGS-HSA-GGGGS-SST14 |
| SEQ ID NO: 10 | SST14-GGGGS-HSA-GGGGS-His6 |
| SEQ ID NO: 11 | HSA-GGGGS-SST14 |
| SEQ ID NO: 12 | SST14-GGGGS-HSA |
| SEQ ID NO: 13 | (SST14-GGGGS)₂-HSA |
| SEQ ID NO: 14 | (SST14-GGGGS)₄-HSA |
| SEQ ID NO: 15 | HSA-(GGGGS)₃-SST14 |
| SEQ ID NO: 16 | HSA-(GGGGS)₆-SST14 |
| SEQ ID NO: 17 | SST-14 |
| SEQ ID NO: 18 | SST-28 |
| SEQ ID NO: 19 | HSA |
| SEQ ID NO: 20 | MDMRVPAQLLGLLLLWLRGARC |
| | (Signal Peptide) |
| SEQ ID NO: 21 | Linker APAPAPAPAPAPAPAPAPAP |
| SEQ ID NO: 22 | Linker APAPAPAPAPAPAPAPAPAPAPAPAPAPA |
| | PAPAPAPAPAP |
| SEQ ID NO: 30 | A(EAAAK)₄A peptide |
| SEQ ID NO: 31 | GGGGS peptide |
| SEQ ID NO: 32 | Linker GGGGSLVPRGSGGGGS |
| SEQ ID NO: 33 | Linker GSGSGS |
| SEQ ID NO: 34 | Linker GGGGSLVPRGSGGGG |
| SEQ ID NO: 35 | Linker GGGGSLVPRGSGGGGS |
| SEQ ID NO: 36 | Linker GGSGGHMGSGG |
| SEQ ID NO: 37 | Linker GGSGGSGGSGG |
| SEQ ID NO: 38 | Linker GGSGGHMGSGG |
| SEQ ID NO: 39 | Linker GGSGG |
| SEQ ID NO: 40 | Linker GGGGSLVPRGSGGGGS |
| SEQ ID NO: 41 | Linker GGSGGGGG |
| SEQ ID NO: 42 | Linker GSGSGSGS |
| SEQ ID NO: 43 | Linker GGGSEGGGSEGGGSEGGG |
| SEQ ID NO: 44 | Linker AAGAATAA |
| SEQ ID NO: 45 | Linker GGGGG |
| SEQ ID NO: 46 | Linker GGSSG |
| SEQ ID NO: 47 | Linker GSGGGTGGGSG |
| SEQ ID NO: 48 | Linker GSGSGSGSGGSGGSGGSGGSGGSGGS |

For the fusion proteins, e.g., SEQ ID NOs: 1-5, 7-10 and 13-16, it should be noted that these are encoded as pro-proteins with a 22 residue signal peptide (SEQ ID NO: 20).

### Somatostatin-Albumin Fusion Proteins

The invention encompasses particles that include polypeptide constructs wherein the somatostatin moiety is encoded by a nucleotide having at least 85% sequence identity to the nucleotide sequence of endogenous human SST-14 or SST-28 (SEQ ID Nos: 23 and 24, respectively).

The invention also encompasses particles that include polypeptide constructs wherein the human serum albumin moiety is encoded by a nucleotide having at least 85% sequence identity to the nucleotide sequence of endogenous human serum albumin (SEQ ID NO: 25). The nucleotide sequence encoding polypeptide constructs can also optionally have at least 90% or 95% sequence identify to SEQ ID NO: 25. The invention further encompasses pharmaceutical compositions that include polypeptide constructs wherein the human serum albumin moiety is a fragment of the endogenous human serum albumin protein, e.g., where it is encoded by a nucleotide consisting of a subsequence of SEQ ID NO: 25. For example, the human serum albumin fragment optionally includes one or more of the three human serum albumin globular domains, each of which contains two subdomains, denominated subdomain IA, IB, IIA, IIB, IIIA, and IIIB (Dockal, 1999, The Journal Of Biological Chemistry, 274(41): 29303-29310).

The invention also encompasses particles that include polypeptide constructs wherein the somatostatin moiety has a polypeptide sequence of at least 85% sequence identity, preferably at least 90% sequence identify, and more preferably at least 95% sequence identity, to the polypeptide sequence of endogenous SST-14 or SST-28 (SEQ ID NOs:17 and 18, respectively).

The invention also encompasses particles that include polypeptide constructs wherein the human serum albumin moiety has a polypeptide sequence at least 85% sequence identity, preferably at least 90% sequence identify, and more preferably at least 95% sequence identity, to the polypeptide sequence of mature human serum albumin (SEQ ID NO: 19).

The invention also encompasses particles that include a fusion protein comprising a signal peptide, a purification tag (His-6), a first linker, a human serum albumin moiety, a second linker and a somatostatin moiety. In one embodiment, the fusion protein is a polypeptide represented by SEQ ID NO: 9 or a sequence having 85% sequence identity to the same.

The invention also encompasses particles that include a fusion protein comprising a somatostatin moiety, a first linker, a human serum albumin moiety, a second linker, a somatostatin moiety and a purification tag (His-6). In one embodiment, the fusion protein is a polypeptide is represented by SEQ ID NO: 10 or a sequence having 85%, 90%, or 95% sequence identity to the same.

The fusion proteins are encoded by a nucleotide sequence (SEQ ID NO: 11) encoding a fusion protein comprising an N-terminal human serum albumin moiety and a C-terminal somatostatin moiety separated by a peptide spacer. Alternatively, the nucleotide sequences alternatively encode an albumin-somatostatin fusion construct which have 85%, 90%, or 95% sequence identity to SEQ ID NO: 11.

The nucleotide sequence (SEQ ID NO: 12) alternatively encodes a fusion protein comprising an N-terminal somatostatin moiety and a C-terminal human serum albumin moiety separated by a peptide spacer. Alternatively, the nucleotide sequences encoding an albumin-somatostatin fusion construct which have 85%, 90%, or 95% sequence identity to SEQ ID NO: 12.

The fusion proteins alternatively include polypeptides wherein the somatostatin moiety comprises two or more copies of the SST-14 or SST-28 sequence arranged in tandem, i.e., “(SST-14)₂” or “(SST-14)₃” or “(SST-28)₂” or “(SST-28)₃”, respectively. Optionally, a linker sequence is included between the two or more tandem somatostatin moieties, and/or a signal peptide sequence is included at the N-terminus of the fusion protein.

The fusion proteins alternatively include polypeptides wherein the somatostatin moiety comprises two or more copies of the SST-14 sequence arranged in a way that at least one copy of the SST14 is linked on both sides by albumin, respectively. Optionally, a linker sequence is included between the two or more tandem somatostatin moieties and between somatostatin and albumin, and/or a signal peptide sequence is included at the N-terminus of the fusion protein. For example, the polypeptide construct may include a signal peptide, two SST-14 moieties separated by a spacer, a second spacer, and an HSA moiety as represented. Optionally, the construct omits the N-terminal signal peptide.

The fusion proteins alternatively include polypeptide constructs wherein the somatostatin moiety comprises two or three copies of the SST-28 sequence arranged in tandem, i.e., “(SST-28)2” or “(SST-28)3”, respectively. Optionally, a linker sequence is included between the two or more tandem somatostatin moieties.

The fusion proteins alternatively include polypeptides comprising any of the albumin-somatostatin fusion proteins described in the preceding paragraphs, where the albumin-somatostatin fusion protein has an in vivo half-life longer than the endogenous SST-14 or SST-28 peptides.

The fusion proteins alternatively include polypeptides comprising any of the albumin-somatostatin fusion proteins described in the preceding paragraphs, wherein the albumin-somatostatin fusion protein has an approximately equal or a greater binding affinity for a somatostatin receptor compared to endogenous SST-14 or SST-28.

The fusion proteins alternatively include polypeptides encompassing albumin-somatostatin fusion proteins comprising an N-terminal albumin moiety as represented by SEQ ID NO: 15, SEQ ID NO: 16, and SEQ ID NO: 2, an internal SST moiety and a C-terminal Albumin moiety as represented by SEQ ID NO: 7 and SEQ ID NO: 8. Optionally, the N-terminus may further include a signal peptide.

Optionally, one or more of the albumin and SST domains may each be separated by an independently selected linker sequence as represented by SEQ ID NO: 1.

In some embodiments, the SST moiety may comprise a pair or plurality of tandem SST sequences, e.g., (SST-14)2 or (SST-28)3, with or without intervening spacing sequences between the two or more tandem SST repeats. Optionally, one or more purification tag sequences may be included in the sequence between two moieties or at the N or C-terminus in order to assist with purification of the fusion protein. An alternative embodiment includes a pair of SST-14 moieties separated by a spacer, as represented by SEQ ID NO: 4. A further embodiment may omit the purification tag (e.g., His6) as shown by the polypeptide sequence represented by SEQ ID NO: 5.

### Somatostatin

The somatostatin domain for use with the fusion proteins of the present invention may be any suitable somatostatin domain, its analogue or derivative. It may be a human somatostatin, any other isolated or naturally occurring somatostatin. The SST moiety can be an analog such as octreotide, lanreotide, pasireotide, seglitide, vapreotide, SST receptor 1 antagonist (e.g. L797=591), SST receptor 2 antagonist (e.g. L779-976), BIM 23014 (octreotide), CH-275 (CAS No. 174688-78-9), SST receptor 3 antagonist (e.g. L796-778), SST receptor 4 antagonist (e.g. L803 087) and/or SST receptor 5 antagonist (e.g. Pasireotide, L817 818).

The fusion proteins may also alternatively include polypeptide constructs wherein the somatostatin moiety comprises a somatostatin analog. Preferably, such an analog is suitable for expression, as part of a fusion protein, in a recombinant host cell. It is understood that a suitable somatostatin analog sequence may be used in place of the SST-14 or SST-28 sequences included in any of the examples disclosed herein.

The fusion proteins alternatively may include polypeptide constructs wherein the somatostatin moiety comprises two or more tandem repeats of a somatostatin polypeptide sequence e.g., SST-14 or SST-28; SEQ ID NOS: 17 and 18, respectively. Each of the repeated somatostatin polypeptide sequences may be a polypeptide sequence having at least 85% sequence identity to SST-14 or SST-28. These repeated variant sequences are independently selected, i.e., in some embodiments the repeats are identical, whereas in other embodiments they are unique.

### Albumin

The albumin for use with the present invention may be any albumin, its analogue or variant. The albumin may be human serum albumin, bovine or equine serum albumin, avian egg albumin, e.g., chicken egg albumin, and/or any other isolated or naturally occurring albumin or fragments thereof.

The fusion proteins alternatively may also include polypeptides wherein the human serum albumin moiety comprises a polypeptide sequence variant with alternative arrangement or number of disulfide bonds due to the presence of additional or fewer cysteine residues than the natural form (e.g., SEQ ID NO: 25).

The albumin also includes different albumin variants, such as proalbumins: Christchurch type (Gainesville, Y-serum 3433), Takefu type, Lille type (Pollibauer, Tokyshima, Taipei); Albumin variants: Nagasaki-3, Yanomama-2, Tagliacozzo, Parklands, Naskapi type (Mersin), Nagasaki-2, Maku, Mexico-2, B, Mi/Fg; Chain termination (Ge/Ct), and etc.

### Spacer or Linker

As described earlier, a spacer or linker can be used with the present invention. The spacer or linker may be independent of the somatostatin or albumin.

The fusion proteins alternatively further include polypeptide constructs wherein the peptide spacer of alternatively referred to as a linker, consists of a polypeptide sequence of from about 2 to about 100 amino acid residues in length. The fusion proteins alternatively encompass polypeptide constructs wherein the peptide spacer is from about 2 to about 50 amino acid residues in length, preferably from about 2 to about from 30, or more preferably from about 3 to about 20 amino acid residues in length.

The fusion proteins alternatively include polypeptide constructs wherein the peptide spacer (alternatively referred to as a linker) has the polypeptide sequence “GGGGS” (SEQ ID NO: 31). Polypeptides rich in Gly, Ser or Thr offer special advantages: (i) rotational freedom of the polypeptide backbone, so that the adjacent domains are free to move relative to on another; (ii) enhanced solubility; (iii) resistance to proteolysis. In addition, many natural linkers exhibited alpha-helical structures. The alpha-helical structure is more rigid and stable than Gly rich linker. An empirical rigid linker with the sequence of A(EAAAK)4A (SEQ ID NO: 30) can be used to separate functional domains. In addition to the role of linking protein domains together, artificial linkers may offer other advantages to the production of fusion proteins, such as improving biological activity, increasing protein expression, and achieving desirable pharmacokinetic profiles.

TABLE 2
A non-exhaustive list of linker sequences
that may be used in the fusion protein
constructs of the present invention.

GGGGSLVPRGSGGGGS (SEQ ID NO: 32)
GSGSGS (SEQ ID NO: 33)
GGGGSLVPRGSGGGG (thrombin proteolytic site
is underlined) (SEQ ID NO: 34)
GGGGSLVPRGSGGGGS (thrombin proteolytic site is
underlined) (SEQ ID NO: 35)
GGSGGHMGSGG (SEQ ID NO: 36)
GGSGGSGGSGG (SEQ ID NO: 37)
GGSGGHMGSGG (SEQ ID NO: 38)
GGSGG (SEQ ID NO: 39)
GGGGSLVPRGSGGGGS (thrombin proteolytic site is
underlined) (SEQ ID NO: 40)
GGSGGGGG (SEQ ID NO: 41)
GSGSGSGS (SEQ ID NO: 42)
GGGSEGGGSEGGGSEGGG (SEQ ID NO: 43)
AAGAATAA (SEQ ID NO: 44)
GGGGG (SEQ ID NO: 45)
GGSSG (SEQ ID NO: 46)
GSGGGTGGGSG (SEQ ID NO: 47)
GT
GSGSGSGSGGSGGSGGSGGSGGSGGS (SEQ ID NO: 48)
GGS
GGGGGGGG (SEQ ID NO: 6)
A(EAAAK)₄A (SEQ ID NO: 20)
APAPAPAPAPAPAPAPAPAP (SEQ ID NO: 21)
APAPAPAPAPAPAPAPAPAPAPAPAPAPAPAPAPAPAPAP
(SEQ ID NO: 22)

### Preparation of Somatostatin-Albumin Fusion Proteins

The somatostatin-albumin fusion proteins employed according to the invention are prepared by expressing a recombinant fusion protein containing the gene encoding introducing the vector into a host. For example, the fusion protein is obtained by expression in a host such as yeast. For example, Pichia pastoris GS115 may be used as a suitable expression host, and the vector used to construct the recombinant expression is pPIC9K. In addition, mammalian lines such as CHO or HEK293 can be used as a preferred expression host.

Plasmid constructs capable of expressing an albumin somatostatin fusion protein comprising a nucleotide sequence encoding a somatostatin albumin fusion protein as described in any of the preceding paragraphs are also provided. For example, suitable plasmid constructs include, but are not limited to, the pcDNA3.1 vector represented by SEQ ID NO: 26 with a DNA sequence encoding any of the albumin-somatostatin fusion proteins disclosed herein ligated into the multiple cloning site of this vector. Other suitable protein expression vectors known in the art may be selected based upon the expression host (e.g., an expression vector with a mammalian promoter system would be suitable for expression in a human cell line whereas a yeast or bacterial expression plasmid would be selected if expression in either of these organisms was desired).

Bacterial or yeast protein expression systems, comprising a bacterial or yeast cell transformed with a plasmid construct comprising a nucleotide sequence that encodes a somatostatin albumin fusion protein are also provided, as described in any of the preceding paragraphs. Suitable bacterial strains include, for example, Escherichia coli. Suitable yeast strains include, for example, Pichia pastoris. An exemplary plasmid construct includes pPIC9K (Invitrogen) as represented by SEQ ID NO: 27, with a nucleotide sequence encoding any of the albumin-somatostatin fusion proteins described herein incorporated into the multiple cloning site of the vector.

Isolated and purified fusion somatostatin fusion proteins are also provided, having a polypeptide sequences as described in any of the preceding paragraphs.

TABLE 3
A list of nucleotide sequences in certain
embodiments of the invention

| | Nucleotide | |
|---|---|---|
| | Sequence Encodes | |
| SEQ ID NO: | the following: | Description |
| SEQ ID NO: 23 | SST14 | Somatostatin-14 |
| | | (SST-14) |
| SEQ ID NO: 24 | SST28 | Somatostatin-28 |
| | | (SST-28) |
| SEQ ID NO: 25 | Human Serum | Human Serum |
| | Albumin | Albumin (HSA) |
| | mature form | |
| SEQ ID NO: 26 | pcDNA3.1(+) | pcDNA3.1(+) |
| | Vector | Vector mammalian |
| | | expression |
| | | vector |
| SEQ ID NO: 27 | pPIC9K Vector | yeast expression |
| | | vector |
| SEQ ID NO: 28 | GGGGS | GGGGS Linker |
| SEQ ID NO: 29 | A(EAAAK)₄A | alpha-helical |
| | | linker |

When the SST is a somatostatin analogue, an alternative method known in the art can be employed to prepare the conjugate. Such alternative method is optionally by chemical synthesis, chemical modification of a peptide, unnatural amino acid incorporation during protein synthesis, and the like.

Eleven SST14-Albumin fusion protein constructs with various linker sequences were designed. Eight of these constructs were made into a fusion gene within a plasmid and produced by HEK 293 transient expression at 100 mL scale. The proteins were collected from the culture media, purified through albumin-based affinity purification, and dialyzed to a storage buffer. These fusion proteins were evaluated for their binding affinity to SSTR2 receptor, and also for cell-based activity in inhibiting cAMP production in a SSTR2-overexpression CHO-K1 cell line. The results of these studies indicated that the length and type of linkers significantly affected the SSTR2 receptor binding affinity, the in-vitro cell-based functional activity, and the fusion protein production yield.

SST-Albumin fusion proteins may have a longer serum half-life and/or more stabilized activity in solution or in a pharmaceutical composition in vitro and/or in vivo compared to the corresponding unfused SST molecules. In rat plasma, for example, more than 90% of SST fusion protein was detected until 40 minutes of incubation, and more than 70% of SST fusion proteins remained up to at least 180 min. Under the same conditions, less than about 50% of free SST remained after 40 minute incubation, and no free SST was detected beyond 120 minutes. Measurement of plasma SST fusion protein concentrations in rats showed that the concentration of the SST fusion protein slowly decreased, with a T_{1/2} of ˜6 hours in contrast to T_{1/2} of several minutes exhibited by plasma concentrations of SST alone. Thus, it would take about 72 hours for the concentration of SST fusion protein to reach zero concentration in plasma.

In addition, SST-Albumin fusion protein exhibited a significantly longer serum half-life and/or improved pharmacokinetic profile in solution or in a pharmaceutical composition in vitro and/or in vivo compared to the corresponding unfused, free SST molecules. The stability of free SST and SST fusion protein was compared in in vitro rat plasma. When incubated in freshly prepared rat plasma at 37° C., free SST and SST fusion protein exhibited degradation half-lives of 33 minutes and 5.5 hours, respectively.

In vivo pharmacokinetic profiles were also generated to demonstrate the improved stability of SST fusion protein relative to free SST. Rats administered intravenously with SST fusion protein exhibited a bi-phasic pharmacokinetic profile, where the α-phase T_{1/2} was 1.01 hour and the β-phase T_{1/2} was 6.14 hour. The calculated half-life is significantly longer than the reported plasma T_{1/2} of free SST in rat (<1 minute; Yogesh C. Patel and Thomas Wheatley. In Vivo and in Vitro Plasma Disappearance and Metabolism of Somatostatin-28 and Somatostatin-14 in the Rat. Endocrinology. Vol. 112, No. 1 (1992), pages 220-225.).

### Preparation and Utility of the Inventive Particles

The inventive particles are therefore contemplated to diagnose or treat any condition for which the encapsulated active agent is known to be effective. For treating cancer, the invention is contemplated to encompass methods of treating cancer in a human subject by administering the inventive particles, as described in any of the preceding paragraphs, wherein the cancer is selected from, for example, breast cancer, colorectal cancer, liver cancer, lung cancer, endocrine cancer, neuroendocrine cancers, pancreatic cancer and prostate cancer.

For example, the particles can be prepared by a method described below.

Particles of albumin fusion proteins can be generated by several different preparation methods, including, but not limited to homogenization, emulsification or chemical crosslinking, by addition of an organic solvent and stabilization at elevated temperature, or diffusion. The particles of albumin fusion proteins can also include other materials as parts of carriers, including but not limited to, biodegradable polymers, non-degradable polymers, lipids, oils and etc. The biodegradable polymers include, but are not limited to, biopolyesters (such as poly(lactic-co-glycolic acid)/PLGA, polylactic acid/PLA, polyglycolic acid/PGA, polycaprolactone/PCL, methoxy poly(ethylene glycol)-block-poly-L-lactide/MPEG-L-PLA, Methoxy Poly(ethylene glycol)-block-poly-DL-lactide/MPEG-DL-PLA, Methoxy Poly(ethylene glycol)-block-poly(ε-caprolactone)/MPEG-PEG-PCL, polyethylene glycol-b-poly {N′-[N-(2-aminoethyl)-2-aminoethyl] aspartamide}/PEG-PAsp (DET), polyhydroxybutyrate), polysaccharides, and proteins. The particles formed of albumin fusion proteins are generally nanoparticles or microspheres, i.e., in with a cross-sectional diameter ranging from about 0.001 μm to about 1 m in diameter for nanoparticles, and 1 μm (1 micron) to about 1000 μm in diameter for microspheres. In one embodiment, this size range is important for the bio-distribution or pharmacokinetic characteristics of these nanoparticles and microspheres. Smaller particles of less than 100-200 nm in diameter, are normally taken up by the reticuloendothelial system (RES) and accumulate in the liver and spleen as well as in solid tumors. Larger particles of from 5-100 μm in diameter, normally target to the capillary bed. When injected into certain tissues, these 5-100 m diameter particles provide a prolonged release, e.g. in the deltoid muscle of the arm, the vastus lateralis muscle of the thigh, the ventrogluteal muscle of the hip, and the dorsogluteal muscle of the buttocks of a patient. The inventive albumin fusion protein microspheres can carry therapeutic agents or diagnostic agents, including both small molecules and macromolecules.

Broadly, the inventive microspheres are prepared by:
1. Fabrication of SST-HSA protein-bound Paclitaxel particles using high pressure homogenization method.

SST-HSA solution is prepared by adding the SST-HSA protein stock solution to deionized (DI) water. 10 mg Paclitaxel is dissolved in Chloroform and added to the SST-HSA solution while homogenizing to form the emulsion with the ratio of SST-HSA protein to Paclitaxel at 10:1 (w/w). Then, transfer the emulsion solution into a rotary evaporator to remove the organic solvent and followed by lyophilization process. The SST-HSA/Paclitaxel powder is then reconstituted in 0.9% saline and further fractioned by filtration. The fractions are used to measure the particle sizes. For example, the preparation produced three fractions having the particle sizes of 86 nm (range 43-122 nm), 164 nm (range 79-190 nm), and 235 nm (range 106-295 nm) in Z-average diameter, as measured by a Malvern Zeta Sizer.
2. Fabrication of SST-HSA/HSA protein-bound Paclitaxel nanoparticles using high pressure homogenization method

A mixture of the SST-HSA/HSA solution is prepared by adding the SST-HSA protein stock solution and HSA with ratios of SST-HSA:HSA=1:9-1:19 in water. Paclitaxel is first dissolved in chloroform and added into the SST-HSA/HSA solution while homogenizing to form emulsion with the 10:1 (w/w) ratio of SST-HSA and HSA to Paclitaxel. Then, transfer the emulsion solution into a rotary evaporator to remove the organic solvent and followed by lyophilization process. The SST-HSA/HSA/Paclitaxel powder is then reconstituted in 0.9% saline to measure the particle sizes. For example, the preparation produced three fractions having Z-average particle sizes at 206 nm (range 78-220 nm) (SST-HAS:HSA=1:9) and 177 nm (range 68-164 nm) (SST-HSA:HSA=1:19) measured by Malvern Zeta Sizer.
3. Fabrication of SST-HSA protein-bound Docetaxel nanoparticles using high pressure homogenization method

SST-HSA solution is prepared by adding SST-HSA protein stock solution to DI water. Docetaxel is dissolved in chloroform. Docetaxel solution is added to the SST-HSA solution while homogenizing to form emulsion. The homogenized emulsion is then transferred into a Rotary evaporator to remove the organic solvent. The final dispersion is then filtered through 0.8 m syringe filter and lyophilized. The reconstituted nanoparticle sizes are measured in 0.9% saline solution. The fractions are used to measure the particle sizes. For example, the preparation produced three fractions having the nanoparticle size is at 113 nm or 0.113 μm (range 68-295 nm) in Z-average diameter measured by Malvern Zeta Sizer.

It is also contemplated to administer the inventive particles to treat cancers that carry somatostatin receptors and/or cancers for which somatostatin analogs are considered to provide effective treatment, e.g., endocrine tumors of the gastrointestinal tract, growth hormone (GH) -secreting pituitary tumors, metastatic endocrine tumors, such as pancreatic tumors and carcinoids. The polymeric shell comprising one or more albumin-somatostatin fusion proteins serves will selectively bind to and target those tumor cells expressing active somatostatin receptors, thus amplifying the selectivity of the particles. Such tumor cells are often inhibited by somatostatin and its analogs. Cancers known to exhibit somatostatin receptors and to respond to SST therapy include, generally, neuroendocrine tumors. For example, these include tumors such as carcinoids, islet-cell carcinoma, glucagonomas, gastrinomas, insulinomas, VIPomas, and medullary thyroid carcinomas. The metabolic basis for this property is thought to be the ability of the neoplastic neuroendocrine cell to incorporate amines intracellularly and to decarboxylate the amines (Kvols, et al, 1992, The Yale Journal of Biology And Medicine 65, 505-518).

The invention also encompasses methods of treating cancer in a human subject by administering a composition containing the fusion protein of the present invention, such as an isolated and purified albumin-somatostatin fusion protein as described in any of the preceding paragraphs. The composition can also include a suitable carrier.

Thus, the invention also encompasses treating somatostatin responsive tumors both with a somatostatin analog in the form of the particle fusion protein, as well as with any anticancer agent that is the payload of the administered nanoparticle.

Any appropriate method can be used to administer a composition that includes the inventive particles. For example, a composition that includes the inventive particles can be administered via injection (e.g., subcutaneous injection, intramuscular injection, intravenous injection, or intrathecal injection or by direct infusion is an organ or potential or actual body cavity).

Before administering a composition that includes the inventive particles to a subject, the subject can be assessed to determine whether or not the subject has a clinical condition, or diagnostic need appropriate for the type of particle to be administered (e.g., a cancer). The artisan can readily determine whether or not a subject should receive such a composition. For example, a subject (e.g., human) can be identified as having a cancer using standard diagnostic techniques.

After identifying a subject as having a clinical condition for which the inventive particles are an appropriate treatment modality, the subject can be administered a composition that includes the appropriate particles.

The invention further encompasses treating somatostatin responsive endocrine cancers, or other types of cancer, by delivering additional anticancer agents, not made a part of the particles, to provide an augmenting or synergistic treatment for such cancers.

An effective amount of a composition that includes inventive particles containing a pharmacologically active ingredient can be any amount that permits a clinically effective result in a subject, wherein the result is one that is appropriate for the pharmacologically active ingredient. When the pharmacologically active ingredient is an anticancer agent, an effective amount is clinically determined by the artisan to be an amount that reduces the progression rate of the cancer, increase the progression-free survival rate, or increase the median time to progression without producing significant toxicity to the subject. Typically, an effective amount of particles that contain and deliver paclitaxel can be from about 50 mg/m² to about 150 mg/m² (e.g., about 80 mg/m²) of the subject. If a particular subject fails to respond to a particular amount, then the amount of particles can be increased by, for example, two or three fold. After receiving this higher concentration, the subject can be monitored for both responsiveness to the treatment and toxicity symptoms, and adjustments made accordingly. The administered amount can remain constant or can be adjusted on a sliding scale or a variable dose depending on the subject's response to treatment. Various factors can influence the actual effective amount used for a particular application. For example, the frequency of administration, duration of treatment, use of multiple treatment agents, route of administration, and severity of the cancer may require an increase or decrease in the actual effective amount administered.

The frequency of administration of the inventive particles can be any frequency that produces or maintains clinical effectiveness. In the instance of an anticancer agent, the frequency is preferably one that reduces the progression rate of the cancer, increases the progression-free survival rate, or increases the median time to progression, without producing significant toxicity to the mammal. For example, the frequency of administration can be from about once a month to about three times a month, or from about twice a month to about six times a month, or from about once every two months to about three times every two months. The frequency of administration can remain constant or can be variable during the duration of treatment. A course of treatment with a composition that includes particles that contain paclitaxel can include rest periods. For example, a composition of particles containing paclitaxel can be administered over a two-week period followed by a two-week rest period, and such a regimen can be repeated multiple times. As with the effective amount, various factors can influence the actual frequency of administration used for a particular application. For example, the effective amount, duration of treatment, use of multiple treatment agents, route of administration, and severity of the cancer may require an increase or decrease in administration frequency.

An effective duration for administering a composition that includes the inventive particles provided herein can be any duration that produces a clinical response. When the particles contain an anticancer agent, the duration is one that reduces the progression rate of cancer, increases the progression-free survival rate, or increases the median time to progression without producing significant toxicity to the subject. Thus, the effective duration can vary from several days to several weeks, months, or years. In general, the effective duration for the treatment of cancer can range in duration from several weeks to several months. In some cases, an effective duration can be for as long as an individual subject is alive. Multiple factors can influence the actual effective duration used for a particular treatment. For example, an effective duration can vary with the frequency of administration, effective amount, use of multiple treatment agents, route of administration, and severity of the cancer.

A composition containing the inventive particles can be in any appropriate form. For example, a composition provided herein can be in the form of a solution or powder, with or without a diluent to make an injectable suspension. A composition can also include additional ingredients including, without limitation, pharmaceutically acceptable vehicles. A pharmaceutically acceptable vehicle can be, for example, saline, water, lactic acid, mannitol, or combinations thereof.

After administering a composition provided herein to a subject, the subject can be monitored to determine whether or not the clinical condition, e.g., cancer was effectively treated. For example, a subject can be assessed after treatment to determine whether or not the progression rate of a cancer was reduced or stopped. As described herein, any art known methods can be used to assess progression and survival rates.

In some cases, a composition that includes inventive particles that contain an anticancer agent can be administered to a subject having cancer under conditions where the 8-week progression-free survival rate for a population of subjects is 65% or greater (e.g., 66%, 67%, 68%, 69%, 70%, 71%, 72%, 73%, 74%, 75%, 76%, 77%, 78%, 79%, 80% or greater) than that observed in a population of comparable subjects not receiving a composition that includes particles containing an anticancer agent. In some cases, a composition that includes particles that contain an anticancer agent can be administered to a subject having cancer under conditions where the median time to progression for a population of subjects is at least 150 days (e.g., at least 155, 160, 163, 165, or 170 days).

An effective amount of a composition that includes inventive particles that contain a diagnostic agent is readily determined by the artisan by administering the composition to a subject and determining if the diagnostic agent is detectable and correlatable with the disease, disorder or condition for which the diagnostic agent is employed.

Biodegradable Polymers, PEGs, phospholipids, and Lipids

In combination with the somatostatin-albumin fusion protein, a number of biocompatible polymers may be employed for the formation of the polymeric shell which surrounds the substantially water insoluble pharmacologically active agents. Essentially any polymer, natural or synthetic, bearing sulfhydryl groups or disulfide bonds within its structure may be utilized for the preparation of a disulfide crosslinked shell about particles of pharmacologically active agents. The sulfhydryl groups or disulfide linkages may be preexisting within the polymer structure or they may be introduced by a suitable chemical modification. For example, natural polymers such as proteins, oligopeptides, polynucleic acids, polysaccharides (e.g., starch, cellulose, dextrans, alginates, chitosan, pectin, hyaluronic acid, and the like), and so on, are candidates for such modification.

As examples of suitable biocompatible polymers, naturally occurring or synthetic proteins may be employed, so long as such proteins have sufficient cysteine residues within their amino acid sequences (i.e. sulfhydryl or disulfide groups) so that crosslinking (through disulfide bond formation, for example, as a result of oxidation during sonication or ultrasonic irradiation) can occur. Examples of suitable proteins include albumin (which contains 35 cysteine residues), insulin (which contains 6 cysteines), hemoglobin (which contains 6 cysteine residues per α₂ β₂ unit), lysozyme (which contains 8 cysteine residues), immunoglobulins, α-2-macroglobulin, fibronectin, vitronectin, fibrinogen, and the like. Other linkages, such as esters, amides, ethers, and the like, can also be formed during the ultrasonic irradiation step (so long as the requisite functional groups are present on the starting material).

The biodegradable polymers that can be incorporated into microspheres and/or nanoparticles, together with albumin fusion proteins include, but are not limited to, biopolyesters (such as poly(lactic-co-glycolic acid)/PLGA, polylactic acid/PLA, polyglycolic acid/PGA, polycaprolactone/PCL, methoxy poly(ethylene glycol)-block-poly-L-lactide/MPEG-L-PLA, Methoxy Poly(ethylene glycol)-block-poly-DL-lactide/MPEG-DL-PLA, Methoxy Poly(ethylene glycol)-block-poly(ε-caprolactone)/MPEG-PEG-PCL, polyethylene glycol-b-poly {N′-[N-(2-aminoethyl)-2-aminoethyl] aspartamide}/PEG-PAsp (DET), polyhydroxybutyrate), polysaccharides, and proteins.

The polymers also include some PEG derivatives, such as monofunctional linear PEGs, bi-functional PEGs, multi-arm PEGs, branched PEGs, heterofunctional PEGs, forked PEGs.

The phospholipids and lipids may also be used in the microspheres and nanoparticles.

The lipids include purified phospholipids from natural sources (such as Hydrogenated soybean phosphatidylcholine/HSPC, Hydrogenated Egg phosphatidylcholine/HEPC, Egg-Sphingomyelin), purified synthetic phospholipids (1,2-Didecanoyl-sn-glycero-3-phosphocholine/DDPC, 1,2-Dilauroyl-sn-glycero-3-phosphocholine/DLPC, 1,2-Dimyristoyl-sn-glycero-3-phosphocholine/DMPC, 1,2-Dipalmitoyl-sn-glycero-3-phosphocholine/DPPC, 1,2-Distearoyl-sn-glycero-3-phosphocholine/DSPC, 1,2-Dilinoleoyl-sn-glycero-3-phosphocholine/DLoPC, 1,2-Dioleoyl-sn-glycero-3-phosphocholine/DOPC, 1,2-Dierucoyl-sn-glycero-3-phosphocholine/DEPC, 1-Myristoyl-2-palmitoyl-sn-glycero-3-phosphocholine/MPPC, 1-Myristoyl-2-stearoyl-sn-glycero-3-phosphocholine/MSPC, 1-Palmitoyl-2-myristoyl-sn-glycero-3-phosphocholine/PMPC, 1-Palmitoyl-2-stearoyl-sn-glycero-3-phosphocholine/PSPC, 1-Myristoyl-2-oleoyl-sn-glycero-3-phosphocholine/MOPC, 1-Palmitoyl-2-oleoyl-sn-glycero-3-phosphocholine/POPC, 1-Stearoyl-2-oleoyl-sn-glycero-3-phosphocholine/SOPC, 1-Myristoyl-2-lyso-sn-glycero-3-phosphocholine/M-LysoPC, 1-Palmitoyl-2-lyso-sn-glycero-3-phosphocholine/P-LysoPC, 1-Stearoyl-2-lyso-sn-glycero-3-phosphocholine/S-LysoPC, 1-Oleoyl-2-lyso-sn-glycero-3-phosphocholine/O-LysoPC, Non-hydrogenated Egg phosphatidylglycerol, sodium salt/EPG-Na, 1,2-Dimyristoyl-sn-glycero-3-phosphoglycerol, sodium salt/DMPG-Na, 1,2-Dimyristoyl-sn-glycero-3-phosphoglycerol, ammonium salt/DMPG-NH₄, 1,2-Dipalmitoyl-sn-glycero-3-phosphoglycerol, sodium salt/DPPG-Na, 1,2-Dipalmitoyl-sn-glycero-3-phosphoglycerol, ammonium salt/DPPG-NH₄, 1,2-Distearoyl-sn-glycero-3-phosphoglycerol, sodium salt/DSPG-Na, 1,2-Distearoyl-sn-glycero-3-phosphoglycerol, ammonium salt/DSPG-NH₄, 1,2-Dioleoyl-sn-glycero-3-phosphoglycerol, sodium salt/DOPG-Na, 1-Palmitoyl-2-oleoyl-sn-glycero-3-phosphoglycerol, sodium salt/POPG-Na, 1,2-Dimyristoyl-sn-glycero-3-phosphatidic acid, sodium salt/DMPA-Na, 1,2-Dipalmitoyl-sn-glycero-3-phosphatidic acid, sodium salt/DPPA-Na, 1,2-Distearoyl-sn-glycero-3-phosphatidic acid, sodium salt/DSPA-Na, Non-hydrogenated Egg phosphatidylethanolamine/EPE, 1,2-Dilauroyl-sn-glycero-3-phosphoethanolamine/DLPE, 1,2-Dimyristoyl-sn-glycero-3-phosphoethanolamine/DMPE, 1,2-Dipalmitoyl-sn-glycero-3-phosphoethanolamine/DPPE, 1,2-Distearoyl-sn-glycero-3-phosphoethanolamine/DSPE, 1,2-Dioleoyl-sn-glycero-3-phosphoethanolamine/DOPE, 1,2-Dilinoleoyl-sn-glycero-3-phosphoethanolamine/DLoPE, 1-Palmitoyl-2-oleoyl-sn-glycero-3-phosphoethanolamine/POPE, 1,2-Dierucoyl-sn-glycero-3-phosphoethanolamine/DEPE, 1,2-Dimyristoyl-sn-glycero-3-phospho-L-serine, sodium salt/DMPS-Na, 1,2-Dipalmitoyl-sn-glycero-3-phospho-L-serine, sodium salt/DPPS-Na, 1,2-Distearoyl-sn-glycero-3-phospho-L-serine, sodium salt/DSPS-Na, 1,2-Dioleoyl-sn-glycero-3-phospho-L-serine, sodium salt/DOPS-Na, 1-Palmitoyl-2-oleoyl-sn-3-phospho-L-serine, sodium salt/POPS-Na,), PEGylated lipids (such as N-(Carbonyl-methoxypolyethyleneglycol 2000)-1,2-distearoyl-sn-glycero-3-phosphoethanolamine, sodium salt/DSPE-PEG 2000, N-(Carbonyl-methoxypolyethyleneglycol 5000)-1,2-distearoyl-sn-glycero-3-phosphoethanolamine, sodium salt/DSPE-PEG 5000, N-(Carbonyl-methoxypolyethyleneglycol 2000)-1,2-dipalmitoyl-sn-glycero-3-phosphoethanolamine, sodium salt/DPPE-PEG 2000, N-(Carbonyl-methoxypolyethyleneglycol 2000)-1,2-dimyristoyl-sn-glycero-3-phosphoethanolamine, sodium salt/DMPE-PEG 2000, 1,2-Distearoyl-sn-glycerol, methoxypolyethylene Glycol/DSG-PEG 5000, 1,2-Distearoyl-sn-glycerol, methoxypolyethylene Glycol/DSG-PEG 2000, 1,2-Dipalmitoyl-sn-glycerol, methoxypolyethylene Glycol/DPG-PEG 2000, 1,2-Dioleoyl-sn-glycerol, methoxypolyethylene Glycol/DOG-PEG 2000, 1,2-Dimyristoyl-sn-glycerol, methoxypolyethylene Glycol/DMG-PEG 2000, N-(Carbonyl-methoxypolyethyleneglycol 2000)-1,2-dipalmitoyl-sn-glycero-3-phosphoethanolamine, sodium salt/DPPE-PEG 5000, N-[Carbonyl-2′,3′-Bis(methoxypolyethyleneglycol 2000)]-1,2-distearoyl-sn-glycero-3-phosphoethanolamine, sodium salt/DSPE-2arm PEG 2000, N-[Carbonyl-2′,3′-Bis(methoxypolyethyleneglycol 5000)]-1,2-distearoyl-sn-glycero-3-phosphoethanolamine, sodium salt/DSPE-2arm PEG 5000, 1,2-Dimyristoyl-sn-glycerol, methoxypolyethylene Glycol/DMG-PEG 5000, 1,2-Dipalmitoyl-sn-glycerol, methoxypolyethylene Glycol/DPG-PEG 5000, 1,2-Dioleoyl-sn-glycerol, methoxypolyethylene Glycol/DOG-PEG 5000), functionalized phospholipids (such as N-(3-Maleimide-1-oxopropyl)-L-α-phosphatidylethanolamine, Dimyristoyl/DMPE-MAL, N-(3-Maleimide-1-oxopropyl)-L-α-phosphatidylethanolamine, Dipalmitoyl/DPPE-MAL, N-(3-Maleimide-1-oxopropyl)-L-α-phosphatidylethanolamine, Distearoyl/DSPE-MAL, N-(3-Maleimide-1-oxopropyl)-L-α-phosphatidylethanolamine, 1-Palmitoyl-2-oleoyl/POPE-MAL, N-(Succinimidyloxy-glutaryl)-L-α-phosphatidylethanolamine, Dioleoyl/DOPE-NHS, N-Glutaryl-L-α-phosphatidylethanolamine, Dimyristoyl/DMPE-Glu, N-Glutaryl-L-α-phosphatidylethanolamine, Dipalmitoyl/DPPE-Glu, N-Glutaryl-L-α-phosphatidylethanolamine, Distearoyl/DSPE-Glu, N-Glutaryl-L-α-phosphatidylethanolamine, Dioleoyl/DOPE-Glu, N-Glutaryl-L-α-phosphatidylethanolamine, 1-Palmitoyl-2-oleoyl/POPE-Glu, N-(aminopropyl polyethyleneglycol)carbamyl-distearoylphosphatidyl-ethanolamine/D SPE-PEG-NH2, N-[(3-Maleimide-1-oxopropyl)aminopropyl polyethyleneglycol-carbamyl] distearoylphosphatidyl-ethanolamine/D SPE-PEG-MAL, 3-(N-succinimidyloxyglutaryl) aminopropyl, polyethyleneglycol-carbamyl di stearoylphosphatidyl-ethanolamine/D SPE-PEG-NHS, N-(3-oxopropoxy polyethyleneglycol)carbamyl-distearoyl-ethanolamine/DSPE-PEG-ALD, 1,2-dipalmitoyl-sn-glycero-3-phosphoethanolamine-N-(7-nitro-2-1,3-b enzoxadiazol-4-yl) [Triethylamine salt]/NBD-DPPE, 1,2-dipalmitoyl-sn-glycero-3-phosphoethanolamine-N-(5-dimethylamino-1-naphthalenesulfonyl) [Triethylamine salt]/Dansyl-DPPE, N-[3-(2-Pyridinyldithio)-1-oxopropyl]-L-α-phosphatidylethanolamine, Dipalmitoyl/DPPE-PDP, N-(Succinimidyloxy-glutaryl)-L-α-phosphatidylethanolamine, Distearoyl/DSPE-NHS, N-(3-Maleimide-1-oxopropyl)-L-α-phosphatidylethanolamine, Dioleoyl/DOPE-MAL, N-(Succinimidyloxy-glutaryl)-L-α-phosphatidylethanolamine, Dimyristoyl/DMPE-NHS, N-(Succinimidyloxy-glutaryl)-L-α-phosphatidylethanolamine, Dipalmitoyl/DPPE-NHS, N-(Succinimidyloxy-glutaryl)-L-α-phosphatidylethanolamine, 1-Palmitoyl-2-oleoyl/POPE-NHS,), novel lipids and cationic lipids (such as 1,2-Dioleoyloxy-3-trimethylammonium propane chloride/DOTAP, 1,2-Dioleyloxy-3-trimethylammonium propane chloride/DOTMA, 1,2-Dioleoyloxy-3-dimethylaminopropane/DODAP, 1,2-Dioleyloxy-3-dimethylaminopropane/DODMA), polyglycin-phospholipids (such as DSPE-polyglycelin-cyclohexyl-carboxylic acid, DSPE-polyglycelin-2-methylglutar-carboxylic acid), SS-cleavable and pH-responsive lipid like materials (such as COATSOME® SS-14/3AP-01, COATSOME® SS-33/3AP-05, COATSOME® SS-33/4PE-15, COATSOME® SS-20/3AP-04), some other excipients (such as PUREBRIGHT® MB series NOFABLE Series SUNBRIGHT® DKH-02HB, DKH-03HB and DKH-04HB (MACROGOL PEG200, 300 and 400)SUNBRIGHT® OE Series (Biocompatible PEG Anchors)).

### Other Components

The polymeric shell according to the invention optionally includes, in addition to the albumin-somatostatin based fusion protein, other components. For example, the polymeric shell includes variable amounts of conventional or variant albumin, or fragments thereof, and preferably, can include any type of human serum albumin.

Optionally, proteins such as α-2-macroglobulin, a known opsonin, are included in the shell composition in order to enhance uptake of the shell encased particles of pharmacologically active agents by macrophage-like cells, or to enhance the uptake of the shell encased particles into the liver and spleen.

Similarly, synthetic polypeptides containing cysteine residues (sulfhydryl or disulfide groups) are also good candidates for formation of a shell on the pharmacologically active agents. In addition, polyalkylene glycols (e.g., linear or branched chain), polyvinyl alcohol, polyhydroxyethyl methacrylate, polyacrylic acid, polyethyloxazoline, polyacrylamide, polyvinyl pyrrolidinone, and the like, are good candidates for chemical modification (to introduce sulfhydryl and/or disulfide linkages) and shell formation (by causing the crosslinking thereof). Thus, for example, contemplated for use in the practice of the present invention are such materials as synthetic polyamino acids containing cysteine residues and/or disulfide groups; polyvinyl alcohol modified to contain free sulfhydryl groups and/or disulfide groups; polyhydroxyethyl methacrylate modified to contain free sulfhydryl groups and/or disulfide groups; polyacrylic acid modified to contain free sulfhydryl groups and/or disulfide groups; polyethyloxazoline modified to contain free sulfhydryl groups and/or disulfide groups; polyacrylamide modified to contain free sulfhydryl groups and/or disulfide groups; polyvinyl pyrrolidinone modified to contain free sulfhydryl groups and/or disulfide groups; polyalkylene glycols modified to contain free sulfhydryl groups and/or disulfide groups; polylactides, polyglycolides, polycaprolactones, or copolymers thereof, modified to contain free sulfhydryl groups and/or disulfide groups; as well as mixtures of any two or more thereof.

Other functional proteins, such as antibodies or enzymes, which facilitate targeting of the pharmaceutical composition to a desired site, are optionally used in the formation of the polymeric shell.

The biocompatible aqueous liquid for carrying or suspending the inventive particles may be selected from, e.g., water, saline, a solution containing appropriate buffers, a solution containing nutritional agents such as amino acids, sugars, proteins, carbohydrates, vitamins or fat, and the like.

### Method of Preparing a Pharmaceutical Composition

In another embodiment, the present invention provides a method for preparing a pharmaceutical composition, including subjecting a mixture containing a pharmacologically active agent and a somatostatin-albumin fusion protein to conditions promoting crosslinking of the somatostatin-albumin fusion protein by disulfide bonds.

The method includes, for example, subjecting a mixture comprising:

an organic phase containing said pharmacologically active agent dispersed therein, and an aqueous medium containing a biocompatible polymer,

wherein said mixture contains surfactants, or optionally, substantially no surfactants, to homogenization in a high pressure homogenizer.

Optionally, the organic and/or aqueous phases are thereafter removed from the mixture after having been subjected to high shear conditions.

Optionally, a dispersing agent to suspend or dissolve the pharmacologically active agent is employed. Dispersing agents contemplated for use in the practice of the present invention include any nonaqueous liquid that is capable of suspending or dissolving the pharmacologically active agent, but does not chemically react with either the polymer employed to produce the shell, or with the pharmacologically active agent itself. Examples include vegetable oils (e.g., soybean oil, mineral oil, corn oil, rapeseed oil, coconut oil, olive oil, safflower oil, cotton seed oil, and the like), aliphatic, cycloaliphatic, or aromatic hydrocarbons having 4-30 carbon atoms (e.g., n-dodecane, n-decane, n-hexane, cyclohexane, toluene, benzene, and the like), aliphatic or aromatic alcohols having 2-30 carbon atoms (e.g., octanol, and the like), aliphatic or aromatic esters having 1-30 carbon atoms (e.g., ethyl caprylate (octanoate), and the like), alkyl, aryl, or cyclic ethers having 2-30 carbon atoms (e.g., diethyl ether, tetrahydrofuran, and the like), alkyl or aryl halides having 1-30 carbon atoms (and optionally more than one halogen substituent, e.g., CH₃ Cl, CH₂ Cl₂, CH₂ Cl—CH₂ Cl, and the like), ketones having 3-30 carbon atoms (e.g., acetone, methyl ethyl ketone, and the like), polyalkylene glycols (e.g., polyethylene glycol, and the like), or combinations of any two or more thereof.

Especially preferred combinations of dispersing agents/organic media typically have a boiling point of no greater than about 200° C., and include volatile liquids such as dichloromethane, chloroform, ethyl acetate, benzene, and the like (i.e., solvents that have a high degree of solubility for the pharmacologically active agent, and are soluble in the other dispersing agent employed), along with a higher molecular weight (less volatile) dispersing agent. When added to the other dispersing agent, these volatile additives help to drive the solubility of the pharmacologically active agent into the dispersing agent. This is desirable, since this step is usually time consuming. Following dissolution, the volatile component may be removed by evaporation (optionally under vacuum).

Particles of pharmacologically active agent, that are substantially contained within a polymeric shell, or associated therewith, prepared as described herein, are delivered neat, or optionally as a suspension in a biocompatible medium. This medium may be selected from water, buffered aqueous media, saline, buffered saline, optionally buffered solutions of amino acids, optionally buffered solutions of proteins, optionally buffered solutions of sugars, optionally buffered solutions of carbohydrates, optionally buffered solutions of vitamins, optionally buffered solutions of synthetic polymers, lipid-containing emulsions, and the like.

In accordance with another embodiment of the present invention, there is provided a method for the preparation of a pharmacologically active agent for in vivo delivery, the method comprising subjecting medium containing a somatostatin-albumin fusion protein and a pharmacologically active agent to high intensity ultrasound conditions for a time sufficient to promote crosslinking of the biocompatible material by disulfide bonds. The pharmacologically active agent can be substantially completely contained within a polymeric shell. The largest cross-sectional dimension of said shell can be no greater than about 10 microns and preferably no greater than about 0.2 microns.

In accordance with another embodiment of the present invention, the somatostatin-albumin fusion protein may be crosslinked as a result of exposure to high shear conditions in a high pressure homogenizer. High shear is used to disperse a dispersing agent, containing dissolved or suspended pharmacologically active agent, into an aqueous solution of the fusion protein, so that a shell of crosslinked polymer is formed around fine droplets of non-aqueous medium. The high shear conditions produce cavitation in the liquid. The cavitation causes local heating and results in the formation of superoxide ions that are capable of crosslinking the fusion protein, for example, by oxidizing the sulfhydryl residues (and/or disrupting existing disulfide bonds) to form new, crosslinking disulfide bonds.

Thus, in accordance with the present invention, a pharmacologically active agent is dissolved in a suitable solvent (e.g., chloroform, methylene chloride, ethyl acetate, ethanol, tetrahydrofuran, dioxane, acetonitrile, acetone, dimethyl sulfoxide, dimethyl formamide, methyl pyrrolidinone, or the like, as well as mixtures of any two or more thereof). Additional solvents contemplated for use in the practice of the present invention include soybean oil, coconut oil, olive oil, safflower oil, cotton seed oil, sesame oil, orange oil, limonene oil, C1-C20 alcohols, C2-C20 esters, C3-C20 ketones, polyethylene glycols, aliphatic hydrocarbons, aromatic hydrocarbons, halogenated hydrocarbons and combinations thereof.

A polymer (e.g. polylactic acid) may not be dissolved in the solvent. The oil phase employed in the preparation of invention compositions can contain only the pharmacologically active agent dissolved in solvent.

Next, a somatostatin-albumin fusion protein is added (into the aqueous phase) to act as a stabilizing agent for the formation of stable nanodroplets. The fusion protein can be added at a concentration in the range of about 0.05 to 25% (w/v), more preferably in the range of about 0.5%-5% (w/v). Surfactant (e.g. sodium lauryl sulfate, lecithin, tween 80, pluronic F-68 and the like) may be added to the mixture.

Next, an emulsion is formed by homogenization under high pressure and high shear forces. Such homogenization is conveniently carried out by forcing the aqueous and oil phase through a homogenizing nozzle at high pressure, using a high pressure homogenizer. A high pressure homogenizer is typically operated at pressures in the range of about 3,000 up to 30,000 psi. Preferably, such processes are carried out at pressures in the range of about 6,000 up to 25,000 psi. The resulting emulsion contains very small nanodroplets of the nonaqueous solvent (containing the dissolved pharmacologically active agent) and very small nanodroplets of the protein stabilizing agent. Acceptable methods of homogenization include processes imparting high shear and cavitation such as high pressure homogenization, high shear mixers, sonication, high shear impellers, and the like.

Finally, the solvent is evaporated under reduced pressure to yield a colloidal system composed of protein coated particles of pharmacologically active agent and the fusion protein. Acceptable methods of evaporation include the use of rotary evaporators, falling film evaporators, spray driers, freeze driers, and the like.

Following evaporation of solvent, the liquid suspension may be dried to obtain a powder containing the pharmacologically active agent and the fusion protein. The resulting powder can be redispersed at any convenient time into a suitable aqueous medium such as saline, buffered saline, water, buffered aqueous media, solutions of amino acids, solutions of vitamins, solutions of carbohydrates, or the like, as well as combinations of any two or more thereof, to obtain a suspension that can be administered to mammals. Methods contemplated for obtaining this powder include freeze-drying, spray drying, and the like.

In accordance with one embodiment of the present invention, there is provided a method for the formation of unusually small submicron particles (nanoparticles), i.e., particles which are less than 0.2 microns in diameter. Such particles are capable of being sterile-filtered before use in the form of a liquid suspension. The ability to sterile-filter the end product of the invention formulation process (i.e., the drug particles) is of great importance since it is impossible to sterilize dispersions which contain high concentrations of protein (e.g., serum albumin) by conventional means such as autoclaving.

In order to obtain sterile-filterable particles (i.e., particles <0.2 microns), the pharmacologically active agent is initially dissolved in a substantially water immiscible organic solvent (e.g., a solvent having less than about 5% solubility in water, such as, for example, chloroform) at high concentration, thereby forming an oil phase containing the pharmacologically active agent. Suitable solvents are set forth above. A polymer (e.g. polylactic acid) may not be dissolved in the solvent. The oil phase employed in the process of the present invention can contain only the pharmacologically active agent dissolved in solvent.

Next, a water miscible organic solvent (e.g., a solvent having greater than about 10% solubility in water, such as, for example, ethanol) is added to the oil phase at a final concentration in the range of about 1%-99% v/v, more preferably in the range of about 5%-25% v/v of the total organic phase. The water miscible organic solvent can be selected from such solvents as ethyl acetate, ethanol, tetrahydrofuran, dioxane, acetonitrile, acetone, dimethyl sulfoxide, dimethyl formamide, methyl pyrrolidinone, and the like. Alternatively, the mixture of water immiscible solvent with the water miscible solvent is prepared first, followed by dissolution of the pharmaceutically active agent in the mixture.

Next, a somatostatin-albumin fusion protein is dissolved in aqueous media. This component acts as a stabilizing agent for the formation of stable nanodroplets. Optionally, a sufficient amount of the first organic solvent (e.g. chloroform) is dissolved in the aqueous phase to bring it close to the saturation concentration. A separate, measured amount of the organic phase (which now contains the pharmacologically active agent, the first organic solvent and the second organic solvent) is added to the saturated aqueous phase, so that the phase fraction of the organic phase is between about 0.5%-15% v/v, and more preferably between 1% and 8% v/v.

Next, a mixture composed of micro and nanodroplets is formed by homogenization at low shear forces. This can be accomplished in a variety of ways, as can readily be identified by those of skill in the art, employing, for example, a conventional laboratory homogenizer operated in the range of about 1,000 up to about 30,000 rpm. This is followed by homogenization under high pressure (i.e., in the range of about 1,000 up to 40,000 psi). The resulting mixture comprises an aqueous protein solution (e.g., human serum albumin), the water insoluble pharmacologically active agent, the first solvent and the second solvent. Finally, solvent is rapidly evaporated under vacuum to yield a colloidal dispersion system (pharmacologically active agent and protein) in the form of extremely small nanoparticles (i.e., particles in the range of about 0.01 microns to 0.2 microns), and thus can be sterile-filtered. The preferred size range of the particles is between about 0.05-0.17 microns, depending on the formulation and operational parameters.

Colloidal systems prepared in accordance with the present invention may be further converted into powder form by removal of the water therefrom, e.g., by lyophilization at a suitable temperature-time profile. The fusion protein itself acts as a cryoprotectant, and the powder is easily reconstituted by addition of water, saline or buffer, without the need to use such conventional cryoprotectants as mannitol, sucrose, glycine, and the like. While not required, it is of course understood that conventional cryoprotectants may be added to invention formulations if so desired.

In accordance with another embodiment of the present invention, a pharmacologically active agent contained within polymeric shells are synthesized using high intensity ultrasound. Two non-linear acoustic processes are involved in the formation of stable polymeric shells (i.e., acoustic emulsification and cavitation). First, acoustic emulsification disperses the pharmacologically active agent into the aqueous protein solution. The dispersion formed is then chemically crosslinked and stabilized by the formation of disulfide bonds. The disulfide bonds are formed from the cysteine residues (in the somatostatin-albumin fusion protein) that are oxidized by superoxide which is produced via acoustic cavitation.

The resulting suspension is optionally filtered through Centricon filters (100 kDa cutoff) and the filtered constructs or microbubbles are resuspended in a normal saline or suitable buffer. The average diameter of these constructs can be approximately 2 microns. Particle size distribution, as determined with a particle counter, can be seen to be quite narrow (a Gaussian distribution with a mean diameter of about 3 microns can be typically observed). The size range of particles obtained by this technique can be 0.1 micron to 20 microns. This size is suited for medical applications, since intravenous or intra-arterial injections can be accomplished without risk of small blood vessel blockage and subsequent tissue (ischemia due to oxygen deprivation) damage. For comparison, normal red blood cells are approximately 8 microns in diameter.

The formation of a shell about the particles of pharmacologically active agent may involve unfolding and reorientation of the somatostatin-albumin fusion protein at the interface between the aqueous and non-aqueous phases such that the hydrophilic regions within the protein are exposed to the aqueous phase while the hydrophobic regions within the protein are oriented towards the non-aqueous phase. In order to effect unfolding of the polymer, or change the conformation thereof, energy must be supplied to the protein. The interfacial free energy (interfacial tension) between the two phases (i.e., aqueous and non-aqueous) contributes to changes in protein conformation at that interface. Thermal energy also contributes to the energy pool required for unfolding and/or change of protein conformation.

Thermal energy input can be a function of such variables as the acoustic power employed in the sonication process, the sonication time, the nature of the material being subjected to sonication, the volume of the material being subjected to sonication, and the like. The acoustic power of sonication processes can vary widely, typically falling in the range of about 1 up to 1000 watts/cm²; with an acoustic power in the range of about 50 up to 200 watts/cm² being a presently preferred range. Similarly, sonication time can vary widely, typically falling in the range of about 2 seconds up to about 5 minutes. Preferably, sonication time will fall in the range of about 15 up to 60 seconds. Those of skill in the art recognize that the higher the acoustic power applied, the less sonication time is required, and vice versa.

The interfacial free energy is directly proportional to the polarity difference between the two phases/liquids. Thus at a given operating temperature a minimum free energy at the interface between the two liquids is essential to form the desired polymeric shell. Thus, if a homologous series of dispersing agents is taken with a gradual change in polarity, e.g., ethyl esters of alkanoic acids, then higher homologues are increasingly nonpolar, i.e., the interfacial tension between these dispersing agents and water increases as the number of carbon atoms in the ester increases. Thus it is found that, although ethyl acetate is water-immiscible (i.e., an ester of a 2 carbon acid), at room temperature (20° C.), this dispersing agent alone will not give a significant yield of polymeric shell-coated particles. In contrast, a higher ester such as ethyl octanoate (ester of an 8 carbon acid) gives polymeric shell-coated particles in high yield. In fact, ethyl heptanoate (ester of a 7 carbon acid) gives a moderate yield while the lower esters (esters of 3, 4, 5, or 6 carbon acids) give poor yield. Thus, at a given temperature, one could set a condition of minimum aqueous-dispersing agent interfacial tension required for formation of high yields of polymeric shell-coated particles.

Temperature is another variable that may be manipulated to affect the yield of polymeric shell-coated particles. In general the surface tension of a liquid decreases with increasing temperature. The rate of change of surface tension with temperature is often different for different liquids. Thus, for example, the interfacial tension (Δy) between two liquids may be Δγ₁ at temperature T₁ and Δγ₂ at temperature T₂. If Δγ₁ at T₁ is close to the minimum required to form the polymeric shells, and if Δγ₂ (at temp. T₂) is greater than Δγ₁, then a change of temperature from T₁ to T₂ will increase the yield of polymeric shells. This can be in the case of ethyl heptanoate, which gives a moderate yield at 20° C., but gives a high yield at 10° C.

Temperature also affects the vapor pressure of the liquids employed. The lower the temperature, the lower the total vapor pressure. The lower the total vapor pressure, the more efficient is the collapse of the cavitation bubble. A more efficient collapse of the sonication bubble correlates with an increased rate of superoxide (H02) formation. Increased rate of superoxide formation leads to increased yields of polymeric shells at lower temperatures. As a countervailing consideration, however, the reaction rate for oxidation of sulfhydryl groups (i.e., to form disulfide linkages) by superoxide ions increases with increasing temperature. Thus for a given liquid subjected to sonication conditions, there exists a fairly narrow range of optimum operating temperatures within which a high yield of polymeric shells is obtained.

Thus a combination of two effects, i.e., the change in surface tension with temperature (which directly affects unfolding and/or conformational changes of the fusion protein) and the change in reaction yield (the reaction being crosslinking of the fusion protein via formation of disulfide linkages) with temperature dictate the overall conversion or yield of polymeric shell-coated particles. Temperatures suitable for the preparation of polymeric shells of the invention can fall in the range of about 0° C.-80° C.

The sonication process described above may be manipulated to produce polymeric shell-coated particles containing pharmacologically active agent having a range of sizes. Presently preferred particle radii fall in the range of about 0.1 up to about 5 microns. A narrow size distribution in this range is very suitable for intravenous drug delivery. The polymeric shell-coated particles are then suspended in an aqueous biocompatible liquid (as described above) prior to administration by suitable means.

In addition, the polymeric shell can optionally be modified by a suitable agent, wherein the agent is associated with the polymeric shell through an optional covalent bond. Covalent bonds contemplated for such linkages include ester, ether, urethane, diester, amide, secondary or tertiary amine, phosphate ester, sulfate ester, and the like bonds. Suitable agents contemplated for this optional modification of the polymeric shell include synthetic polymers (polyalkylene glycols (e.g., linear or branched chain polyethylene glycol), polyvinyl alcohol, polyhydroxyethyl methacrylate, polyacrylic acid, polyethyloxazoline, polyacrylamide, polyvinyl pyrrolidinone, and the like), phospholipids (such as phosphatidyl choline (PC), phosphatidyl ethanolamine (PE), phosphatidyl inositol (PI), sphingomyelin, and the like), proteins (such as enzymes, antibodies, and the like), polysaccharides (such as starch, cellulose, dextrans, alginates, chitosan, pectin, hyaluronic acid, and the like), chemical modifying agents (such as pyridoxal 5′-phosphate, derivatives of pyridoxal, dialdehydes, diaspirin esters, and the like), or combinations of any two or more thereof.

Variations on the general theme of dissolved pharmacologically active agent enclosed within a polymeric shell are possible. A suspension of fine particles of pharmacologically active agent in a biocompatible dispersing agent could be used (in place of a biocompatible dispersing agent containing dissolved pharmacologically active agent) to produce a polymeric shell containing dispersing agent-suspended pharmacologically active agent particles. In other words, the polymeric shell could contain a saturated solution of pharmacologically active agent in dispersing agent. Another variation is a polymeric shell containing a solid core of pharmacologically active agent produced by initially dissolving the pharmacologically active agent in a volatile organic solvent (e.g. benzene), forming the polymeric shell and evaporating the volatile solvent under vacuum, e.g., in a rotary evaporator, or freeze-drying the entire suspension. This results in a structure having a solid core of pharmacologically active agent surrounded by a polymer coat. This latter method is particularly advantageous for delivering high doses of pharmacologically active agent in a relatively small volume. In some cases, the polymer forming the shell about the core could itself be a therapeutic or diagnostic agent, e.g., in the case of insulin, which may be delivered as part of a polymeric shell formed in the sonication process described above.

Variations in the polymeric shell are also possible. For example, a small amount of PEG containing sulfhydryl groups could be included with the fusion protein. Upon sonication, the PEG is crosslinked into the fusion protein and forms a component of the polymeric shell. Alternatively, PEG can be linked to the polymeric shell following the preparation of the shell (rather than being included as part of the media from which the shell is prepared). PEG is known for its nonadhesive character and has been attached to proteins and enzymes to increase their circulation time in vivo [Abuchowski et al., J. Biol. Chem. Vol. 252:3578 (1977)]. It has also been attached to phospholipids forming the lipidic bilayer in liposomes to reduce their uptake and prolong lifetimes in vivo [Klibanov et al., FEBS Letters Vol. 268:235 (1990)]. Thus the incorporation of PEG into the walls of crosslinked protein shells alters their blood circulation time. This property can be exploited to maintain higher blood levels of the pharmacologically active agent and prolonged pharmacologically active agent release times.

Electrophilic PEG derivatives including PEG-imidazoles, succinimidyl succinates, nitrophenyl carbonates, tresylates, and the like; nucleophilic PEG derivatives including PEG-amines, amino acid esters, hydrazides, thiols, and the like are also useful for the modification of the polymeric shell. The PEG-modified polymeric shell will persist in the circulation for longer periods than their unmodified counterparts. The modification of a polymeric shell with PEG may be performed before formation of the shell, or following formation thereof. The currently preferred technique is to modify the polymeric shell after formation thereof. Other polymers including dextran, alginates, hydroxyethyl starch, and the like, may be utilized in the modification of the polymeric shell.

One skilled in the art will recognize that several variations are possible within the scope and spirit of this invention. The dispersing agent within the polymeric shell may be varied, a large variety of pharmacologically active agents may be utilized, and a wide range of proteins as well as other natural and synthetic polymers may be used in the formation of the walls of the polymeric shell. Applications are also fairly wide ranging.

In accordance with yet another embodiment of the present invention, the above-described mode of administration is facilitated by novel docetaxel-containing compositions in which docetaxel is suspended in a biocompatible liquid, and wherein the resulting suspension contains particles of docetaxel having a cross-sectional dimension no greater than 10 microns and preferably 0.2 microns. The desired particle size of less than about 10 microns can be achieved in a variety of ways, e.g., by grinding, spray drying, precipitation, sonication, and the like.

The particles of docetaxel preferably have size less than 10 microns, more preferably less than 5 microns and most preferably less than 1 micron, which allows intravenous delivery in the form of a suspension without the risk of blockage in the microcirculation of organs and tissues.

Due to the nanoparticle nature of the delivered drug, most of it is cleared from the circulation by organs having reticuloendothelial systems such as the spleen, liver, and lungs. This allows pharmacologically active agents in particulate form to be targeted to such sites within the body.

Biocompatible liquids contemplated for use in this embodiment are the same as those described above. In addition, parenteral nutritional agents such as Intralipid (trade name for a commercially available fat emulsion used as a parenteral nutrition agent; available from Kabi Vitrum, Inc., Clayton, N.C.), Nutralipid™ (trade name for a commercially available fat emulsion used as a parenteral nutrition agent; available from McGaw, Irvine, Calif.), Liposyn III (trade name for a commercially available fat emulsion used as a parenteral nutrition agent (containing 20% soybean oil, 1.2% egg phosphatides, and 2.5% glycerin); available from Abbott Laboratories, North Chicago, Ill.), and the like may be used as the carrier of the drug particles. Alternatively, if the biocompatible liquid contains a drug-solubilizing material such as soybean oil (e.g., as in the case of Intralipid™), the drug may be partially or completely solubilized within the carrier liquid, aiding its delivery. An example of such a case is the delivery of docetaxel in Intralipid™ as the carrier. Presently preferred biocompatible liquids for use in this embodiment are parenteral nutrition agents, such as those described above.

In another embodiment, the present invention provides a method for treating cancer in a subject, including a human subject, by administering the pharmaceutical composition containing the pharmacologically active agent and the polymeric shell.

In accordance with still another embodiment of the present invention, there is provided a composition for the in vivo delivery of docetaxel wherein docetaxel is dissolved in a parenteral nutrition agent.

### EXAMPLES

Selected embodiments of the invention will be described in further detail with reference to the following experimental and comparative examples. These examples are for illustrative purposes only and are not intended to limit the scope of the invention.

### Example 1: Expression of Fusion Proteins in Mammalian Systems

### Example 1-1. Recombinant Gene Synthesis

Eight constructs corresponding to the fusion proteins listed in Table 5 were prepared. First, the gene sequence coding each fusion protein was de novo synthesized and subsequently inserted into the pcDNA3.1 vector.

### Example 1-2. Plasmid Generation

Maxi-prep or Mega-prep was used to generate ˜20 mg of each DNA.

### Example 1-3. Transfection and Protein Production

(A) Suspension Cell Method

FreeStyle™ 293-F Cells were seeded at 0.55-0.6×10⁶ cells/mL in a flask. After about 24 hours, the cells were seeded in a shake flask at 1.1-1.2×10⁶ cells/mL. DNA was prepared at 500 ug DNA/80 mL in a FreeStyle medium. Polyethylenimine (PEI) was prepared at 1.8 mL PEI per 80 mL in a FreeStyle medium. DNA was mixed in the FreeStyle medium, and the effective amount of PEI was added to the DNA solution, and the mixture is vortexed incubated for about 15 minutes at room temperature to form DNA-PEI complex. An 80 mL of the incubated DNA-PEI complex is added to a cell culture. About 3 hours later, TC Yeastolate feed (BD) is added to have the final concentration of 4 gram/liter of culture. After about 7-8 days, the medium is harvested by centrifugation.

(B) Adherent Cell Method

About 24 hours before transfection, HEK293 cells were seeded to 50-90% confluency in a flask, and complete medium is added. After about 24 hours, cells were washed followed by adding basal medium.

DNA and PEI solutions are prepared by adding DNA to a serum free medium. The PEI solution was added to the DNA solution and incubated for 15 minutes to form DNA-PEI complex at room temperature.

The DNA-PEI complex was added to cells, and the mixture incubated for about 4-6 hours at 37° C. The medium was removed and fresh medium with Glutamine and serum was added, followed by incubating at 37° C. for 4 days.

The medium was harvested after about 4 days, by centrifuging to collect the supernatant. The precipitate was replenished with fresh medium with L-Glutamine for another 3-day incubation to repeat the harvesting process.

### Example 1-4. Protein Concentration, Ni-NTA Purification and Buffer Exchange

The collected medium was concentrated by TFF system (Millipore) to a certain volume depending on purification methods (either continuous chromatography or manual batch purification).

The concentrated proteins were incubated with fresh Ni-NTA resin at about 4° C. in binding buffer and washed with wash buffer using either chromatography or batch system. The protein was eluted with elute buffer and fractions were collected and concentrated to recover the purified protein. The protein can be further purified using size exclusion chromatography purification.

The buffer of the final eluate can be exchanged by dialysis to a desired buffer.

### Example 2: Yields of Sst-Albumin Fusion Proteins

The SST-HSA fusion proteins were all expressed in soluble form with high yield. The length or the nature of the linkers can affect the protein yield and solubility of the fusion proteins. The results indicated that the production yield slightly decreased as the fusion protein constructs became longer and more complex. However, all the constructs exhibited yield for scale up production.

TABLE 4
SST14-HSA fusion protein expression yield

| | | Total | | Production |
|---|---|---|---|---|
| | | amino | MW | Yield |
| Sequence ID | Design | acids | (kDa) | (g/L) |
| SEQ ID NO: 1 | SST14-A(EAAAK)₄A-HSA- | 657 | 73.8364 | 0.26 |
| | A(EAAAK)₄A-SST14 | | | |
| SEQ ID NO: 2 | HSA A(EAAAK)₄A-SST14 | 621 | 70.1543 | 0.27 |
| SEQ ID NO: 7 | SST14-(GGGGS)₃-HSA | 614 | 69.112 | 0.33 |
| SEQ ID NO: 8 | SST14-A(EAAAK)₄A-HSA | 621 | 70.1543 | 0.25 |
| SEQ ID NO: 9 | H6-GGGGS-HSA-GGGGS- | 613 | 69.4874 | 0.30 |
| | SST14 | | | |
| SEQ ID NO: 10 | SST14-GGGGS-HSA-GGGGS- | 613 | 69.4874 | 0.41 |
| | His6 | | | |
| SEQ ID NO: 15 | HSA-(GGGGS)₃-SST14 | 614 | 69.112 | 0.28 |
| SEQ ID NO: 16 | HSA-(GGGGS)₆-SST14 | 629 | 70.1119 | 0.29 |

### Example 3: Preparation of Docetaxel Particles in Aqueous Medium

Crystals of docetaxel are ground in a ball mill until particles of solid docetaxel are obtained having a size less than 10 microns. Size of particles are determined by suspending the particles in isotonic saline and counting with the aid of a particle counter. Grinding is continued until 100% of the particles had a size less than 5 microns. The preferred particle size for intravenous delivery is less than 5 microns and most preferably less than 1 micron.

Alternatively, particles of docetaxel are obtained by sonicating a suspension of docetaxel in water until all particles are below 10 microns in diameter.

Docetaxel particles less than 10 microns in diameter can also be obtained by precipitating docetaxel from a solution of docetaxel in ethanol by adding water until a cloudy suspension is obtained. Optionally, the solution of docetaxel can be sonicated during the water addition, until a cloudy suspension is obtained. The resulting suspension is then filtered and dried to obtain pure docetaxel particles in the desired size range.

Fine particles of docetaxel are prepared by spray drying a solution of docetaxel in a volatile solvent such as ethanol. The solution is passed through an ultrasonic nozzle that forms droplets of ethanol containing docetaxel. As the ethanol evaporated in the spray drier, fine particles of docetaxel are obtained. Particle size is varied by changing the concentration of docetaxel in ethanol, adjusting the flow rate of liquid through the nozzle and power of sonication. Suitable sonicators include Vibracell VCX 750 with model CV33 probe head, Sonics and Materials Inc., Newtown, Conn.

### Example 4: Preparation of Protein Shell Containing Oil

Three ml of a 5% somatostatin-albumin fusion protein solution are taken in a cylindrical vessel that is attached to a sonicating probe. Suitable sonicators include Vibracell VCX 750 with model CV33 probe head, Sonics and Materials Inc., Newtown, Conn. The somatostatin-albumin fusion protein solution is overlayered with 6.5 ml of soybean oil (soya oil). The tip of the sonicator probe is brought to the interface between the two solutions and the assembly is maintained in a cooling bath at 20° C. The system is allowed to equilibrate and the sonicator is turned on for 30 seconds. Vigorous mixing occurs and a white milky suspension is obtained. The suspension is diluted 1:5 with normal saline. A particle counter is utilized to determine size distribution and concentration of oil-containing protein shells.

### Example 6: Parameters Affecting Polymeric Shell Formation

Several variables such as protein concentration, temperature, sonication time, concentration of pharmacologically active agent, and acoustic intensity are tested to optimize formation of polymeric shell. These parameters are determined for crosslinked somatostatin-albumin fusion protein shells containing toluene.

Polymeric shells made from solutions having protein concentrations of 1%, 2.5%, 5% and 10% are counted with the particle counter to determine a change in the size and number of polymeric shells produced. The size of the polymeric shells varies with protein concentration, but the number of polymeric shells per milliliter of “milky suspension” formed increases with the increase in concentration of the protein up to 5%. No significant change in the number of polymeric shells occurs above that concentration.

Initial vessel temperatures are important for optimal preparation of polymeric shells. Typically, initial vessel temperatures are maintained between 0° C. and 45° C. The aqueous-oil interfacial tension of the oils used for formation of the polymeric shell is an important parameter, which also varies as a function of temperature. The concentration of pharmacologically active agent does not significantly affect the yield of protein shells. It is relatively unimportant if the pharmacologically active agent is incorporated in the dissolved state, or suspended in the dispersing medium.

Sonication time is an important factor determining the number of polymeric shells produced per ml. A sonication time greater than three minutes produces a decrease in the overall count of polymeric shells, indicating possible destruction of polymeric shells due to excessive sonication. Sonication times less than three minutes produce adequate numbers of polymeric shells. Regarding the acoustic power rating of the sonicator, the maximum number of polymeric shells are produced at the highest power setting, e.g., with an acoustic power at about 200 watts/cm².

### Example 7: Preparation of Polymeric Shells Containing Docetaxel in Oil Carrier

Docetaxel is dissolved in USP grade soybean oil at a concentration of 2 mg/ml. 3 ml of a 5% somatostatin-albumin fusion protein solution is taken in a cylindrical vessel that could be attached to a sonicating probe. The somatostatin-albumin fusion protein solution is overlayered with 6.5 ml of soybean oil/docetaxel solution. The tip of the sonicator probe is brought to the interface between the two solutions and the assembly is maintained in equilibrium and the sonicator turns on for 30 seconds. Vigorous mixing occurs and a stable white milky suspension is obtained which contains protein-walled polymeric shells enclosing the oil/docetaxel solution.

In order to obtain a higher loading of drug into the crosslinked protein shell, a mutual solvent for the oil and the drug (in which the drug has a considerably higher solubility) can be mixed with the oil. Provided this solvent is relatively non-toxic (e.g., ethyl acetate), it may be injected along with the original carrier. In other cases, it may be removed by evaporation of the liquid under vacuum following preparation of the polymeric shells.

### Example 8: Stability of Polymeric Shells

Suspensions of polymeric shells at a known concentration are analyzed for stability at three different temperatures (i.e., 4° C., 25° C., and 38° C.). Stability is measured by the change in particle counts over time. Crosslinked protein (somatostatin-albumin fusion protein) shells containing soybean oil (SBO) are prepared as described above (see Example 2), diluted in saline to a final oil concentration of 20% and stored at the above temperatures. Particle counts (Elzone) are obtained for each of the samples as a function of time.

The concentration of counted particles (i.e., polymeric shells) remains fairly constant over the duration of the experiment. The range indicates good polymeric shell stability under a variety of temperature conditions over almost four weeks.

### Example 9: In Vivo Biodistribution—Crosslinked Protein Shells Containing A Fluorophore

To determine the fate of crosslinked somatostatin-albumin fusion protein shells following intravenous injection, a fluorescent dye (rubrene, a/k/a (5,6,11,12-tetraphenyltetracene, obtained from Aldrich) is dissolved in toluene, and crosslinked protein shells containing toluene/rubrene are prepared as described above by sonication. The resulting milky suspension is diluted five times in normal saline. Two ml of the diluted suspension is then injected into the tail vein of a rat over 10 minutes. One animal is sacrificed an hour after injection and another 24 hours after injection.

Frozen lung, liver, kidney, spleen, and bone marrow sections are examined under fluorescence for the presence of polymeric shells containing fluorescent dye. At one hour, most of the polymeric shells are intact and found in the lungs and liver as brightly fluorescing particles of about 1 micron diameter. At 24 hours, polymeric shells are found in the liver, lungs, spleen, and bone marrow. A general staining of the tissue is also observed, indicating that the polymeric shells are digested, and the dye liberates from within. This result is consistent with expectations and demonstrates the potential use of invention compositions for delayed or controlled release of entrapped pharmaceutical agent such as docetaxel.

### Example 10: Toxicity of Polymeric Shells Containing Soybean Oil (SBO)

Polymeric shells containing soybean oil (SBO) are prepared as described in Example 2. The resulting suspension is diluted in normal saline to produce two different solutions, one containing 20% SBO and the other containing 30% SBO.

Intralipid™, a commercially available total parenteral nutrition (TPN) agent, contains 20% SBO. The LD₅₀ for Intralipid™ in mice is 120 ml/kg, or about 4 ml for a 30 g mouse, when injected at 1 cc/min.

Two groups of mice (three mice in each group; each mouse weighing about 30 g) are treated with invention composition containing SBO as follows. Each mouse is injected with 4 ml of the prepared suspension of SBO-containing polymeric shells. Each member of one group receives the suspension containing 20% SBO, while each member of the other group receives the suspension containing 30% SBO.

The oil contained within polymeric shells according to the present invention is not toxic at its LD₅₀ dose, as compared to a commercially available SBO formulation (Intralipid™). This effect can be attributed to the slow, such as one or more hours, release (i.e., controlled rate of becoming bioavailable) of the oil from within the polymeric shell. Such slow release prevents the attainment of a lethal dose of oil, in contrast to the high oil dosages attained with commercially available emulsions.

### Example 11: In Vivo Bioavailability of Soybean Oil Released from Polymeric Shells

A test is performed to determine the slow or sustained release of polymeric shell-enclosed material following the injection of a suspension of polymeric shells into the blood stream of rats. Crosslinked protein (somatostatin-albumin fusion protein) walled polymeric shells containing soybean oil (SBO) are prepared by sonication as described above. The resulting suspension of oil-containing polymeric shells is diluted in saline to a final suspension containing 20% oil. Five ml of this suspension is injected into the cannulated external jugular vein of rats over a 10 minute period. Blood is collected from these rats at several time points following the injection and the level of triglycerides (soybean oil is predominantly triglyceride) in the blood determined by routine analysis.

Five milliliter of a commercially available fat emulsion (Intralipid™, an aqueous parenteral nutrition agent—containing 20% soybean oil, 1.2% egg yolk phospholipids, and 2.25% glycerin) is used as a control. The control utilizes egg phosphatide as an emulsifier to stabilize the emulsion. A comparison of serum levels of the triglycerides in the two cases would give a direct comparison of the bioavailability of the oil as a function of time. In addition to the suspension of polymeric shells containing 20% oil, five ml of a sample of oil-containing polymeric shells in saline at a final concentration of 30% oil is also injected. Two rats are used in each of the three groups.

For the Intralipid™ control, very high triglyceride levels are seen following injection. Triglyceride levels are then seen to take about 24 hours to come down to preinjection levels. Thus the oil is seen to be immediately available for metabolism following injection.

The suspension of oil-containing polymeric shells containing the same amount of total oil as Intralipid™ (20%) show a dramatically different availability of detectible triglyceride in the serum. The level indicates a slow or sustained release of triglyceride into the blood at levels fairly close to normal. The group receiving oil-containing polymeric shells having 30% oil shows a higher level of triglycerides (concomitant with the higher administered dose). Once again, the blood levels of triglyceride do not rise astronomically in this group, compared to the control group receiving Intralipid™. This again, indicates the slow and sustained availability of the oil from invention composition, which has the advantages of avoiding dangerously high blood levels of material contained within the polymeric shells and availability over an extended period at acceptable levels. Clearly, drugs delivered within polymeric shells of the present invention would achieve these same advantages.

Such a system of soybean oil-containing polymeric shells could be suspended in an aqueous solution of amino acids, essential electrolytes, vitamins, and sugars to form a total parenteral nutrition (TPN) agent. Such a TPN cannot be formulated from currently available fat emulsions (e.g., Intralipid™) due to the instability of the emulsion in the presence of electrolytes.

### Example 12: Preparation of Crosslinked Protein-Walled Polymeric Shells Containing a Solid Core of Pharmaceutically Active Agent

Another method of delivering a poorly water-soluble drug such as docetaxel within a polymeric shell is to prepare a shell of polymeric material around a solid drug core. Such a ‘protein coated’ drug particle may be obtained as follows. The procedure described in Example 4 is repeated using an organic solvent to dissolve docetaxel at a relatively high concentration.

Solvents generally used are organics such as benzene, toluene, hexane, ethyl ether, and the like.

Polymeric shells are produced as described in Example 4. 5 mL of the milky suspension of polymeric shells containing dissolved docetaxel are diluted to 10 ml in normal saline. This suspension is placed in a rotary evaporator at room temperature and the volatile organic removed by vacuum. After about 2 hours in the rotary evaporator, these polymeric shells are examined under a microscope to reveal opaque cores, indicating removal of substantially all organic solvent, and the presence of solid docetaxel within a shell of protein.

Alternatively, the polymeric shells with cores of organic solvent-containing dissolved drug are freeze-dried to obtain a dry crumbly powder that can be resuspended in saline (or other suitable liquid) at the time of use. In case of other drugs that may not be in the solid phase at room temperature, a liquid core polymeric shell is obtained. This method allows for the preparation of a crosslinked protein-walled shell containing undiluted drug within it. Particle size analysis shows these polymeric shells to be smaller than those containing oil. Although the presently preferred protein for use in the formation of the polymeric shell is somatostatin-albumin fusion protein, other proteins such as α-2-macroglobulin, a known opsonin, could be used to enhance uptake of the polymeric shells by macrophage-like cells. Alternatively, a PEG-sulfhydryl (described below) could be added during formation of the polymeric shell to produce a polymeric shell with increased circulation time in vivo.

### Example 13: In Vivo Circulation and Release Kinetics of Polymeric Shells

Solid core polymeric shells containing docetaxel are prepared as described above (see, for example, Example 4) and suspended in normal saline. The concentration of docetaxel in the suspension is measured by HPLC as follows. First, the docetaxel within the polymeric shell is liberated by the addition of 0.1M mercaptoethanol (resulting in exchange of protein disulfide crosslinkages, and breakdown of the crosslinking of the polymeric shell), then the liberated docetaxel is extracted from the suspension with acetonitrile. The resulting mixture is centrifuged and the supernatant is freeze-dried. The lyophilate is dissolved in methanol and injected onto an HPLC to determine the concentration of docetaxel in the suspension.

Rats are injected with 2 ml of this suspension through a jugular catheter. The animal is sacrificed at two hours, and the amount of docetaxel present in the liver is determined by HPLC. This requires homogenization of the liver, followed by extraction with acetonitrile and lyophilization of the supernatant following centrifugation. The lyophilate is dissolved in methanol and injected onto an HPLC.

### Example 14: Composition, Preparation, and Drug Release of Sst-Hsa Paclitaxel Nanoparticles

### Example 14-1: Fabrication of SST-HSA Protein-Bound Paclitaxel Nanoparticles Using High Pressure Homogenization Method

10 ml of 1% (w/v) SST-fusion proteins and HSA solution was prepared by adding 4 ml of 2.5 mg/ml SST-HSA protein and 360 μl of 25% HSA to 5.64 ml of DI water, and then the solution was pre-saturated with 52 μl of Chloroform. 10 mg of paclitaxel was dissolved in a mixture of 183 μl of chloroform and 17 μl of Ethanol. The paclitaxel solution was added to the 10 ml SST-fusion proteins and HSA solution with homogenizing. The mixture was homogenized for additional few minutes to form a crude emulsion. The crude emulsion was transferred into a high pressure homogenizer. The emulsification was performed at high pressure while recycling the emulsion for few minutes. The homogenized emulsion was concentrated by removing the volatile organic solvent using a rotary evaporator, followed by removing the rest of the solvent by lyophilization to obtain nanoparticles. The final nanoparticles were stored at 4° C. The size of reconstituted nanoparticles was measured using a Malvern Zeta Sizer, and the Z-average particle size of the resulting the particles were between 0.17 to 0.2 microns (170 to 200 nm). The nanoparticles were further fractioned by filtration or other methods to collect three fractions of nanoparticles, about less than 0.1 microns (100 nm), 0.1 to 0.2 microns (100-200 nm), and over 0.2 microns (200 nm) in diameter. Suitable homogenizers include an in-line Megatron homogenizer MT-V 3-65 F/FF/FF, Kinematica AG, Switzerland.

Another batch of nanoparticles were prepared with 2 ml of 2.5 mg/ml SST-HSA protein and 360 ml of 25% HSA in 7.64 ml of DI water. The size distribution of this batch of nanoparticles was similar to the above nanoparticles.

### Example 14-2: Release of SST Protein-Bound Paclitaxel Nanoparticles In Vitro

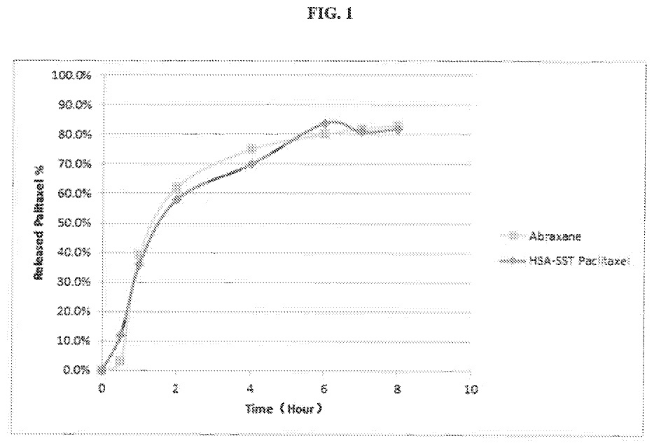

In-vitro release studies was performed with SST-HSA Paclitaxel nanoparticles using USP Apparatus II. The Abraxane sample was also used as a reference. The samples were taken periodically over 8 hours and paclitaxel concentration is measured by HPLC. The release profiles of SST-HSA paclitaxel nanoparticles and Abraxane nanoparticles are shown in FIG. 1:

The result indicated that SST-fusion protein did not alter the release profile of the paclitaxel.

### Example 15: Cytotoxicity of Paclitaxel-Conjugated Complexes

Cytotoxicity of a Paclitaxel-SST fusion protein (SEQ ID NO: 1). Conjugated Complex has been tested in CHO cells expressing human recombinant sst2a. Cell viability was measured using the CellTiter-Glo® Luminescent Cell Viability Assay (Promega) that determines the number of viable cells in culture, based on quantitation of the ATP present that is an indicator of metabolically active cells. The assay was conducted according to the manufacturer's manual (Promega Technical Bulletin, Part # TB288, for products G7570, G7571, G7572 and G7573, revised June 2009).

Digitonin at 20 μg/ml concentration was used as positive controls, DMSO-treated and agonist (Octreotide)-treated wells were used as vehicle controls. After dispensing test compounds to the wells, cells in the incubation buffer were added to the wells and incubated for 20 minutes. At the end of incubation, CellTiter-Glo was added to each well to measure luminescence. All testing wells contained 0.4% DMSO. No significant cytotoxicity was observed at various paclitaxel concentrations of 0.14 nM, 0.42 nM, 1.4 nM, 4.2 nM, 14 nM, 42 nM, and 110 nM (Table 5).

The result indicated that SST-fusion protein is safe to be employed as a drug-delivery protein.

TABLE 5
Cytotoxicity of Paclitaxel-Conjugated Compounds

| | | | | Cytotoxicity | Standard |
|---|---|---|---|---|---|
| | Compounds | Conc. | Unit | % | Deviation |
| test 1 | Digitonin | 20 | mg/ml | 100.0 | NA |
| | (Positive | | | | |
| | Control) | | | | |
| | DMSO | 0.4 | % | 0.0 | NA |
| | (Vehicle only) | | | | |
| | Octreotide | 10 | nM | −4.0 | 3.2 |
| | (Agonist) | | | | |
| | Paclitaxel-SST | 0.14 | nM | 3.0 | 9.1 |
| | fusion protein | 0.42 | nM | 12.0 | 6.8 |
| | Conjugated | 1.4 | nM | 1.0 | 3.3 |
| | Complex | 4.2 | nM | −5.0 | 14.1 |
| | | 14.0 | nM | −1.0 | 2.7 |
| | | 42.0 | nM | 0.0 | 9.1 |
| Test 2 | Digitonin | 20 | mg/ml | 100.0 | NA |
| | (Positive | | | | |
| | Control) | | | | |
| | DMSO | 0.4 | % | 0.0 | NA |
| | (Vehicle only) | | | | |
| | Octreotide | 10 | nM | 8.0 | 1.9 |
| | (Agonist) | | | | |
| | Paclitaxel-SST | 110 | nM | 11.0 | 4 |
| | fusion protein | | | | |
| | Conjugated | | | | |
| | Complex | | | | |

### Example 16: Inhibition of SST Binding onto SST2A Receptor by Paclitaxel SST-Fusion Protein (SEQ ID NO: 1) Conjugated Complex

Various concentration of 0.3 nM, 1 nM, 3 nM, 10 nM, 0.03 μM and 0.1 μM of paclitaxel SST-fusion protein (SEQ ID NO: 1) were tested in SST and sst2 receptor binding inhibition assay. Paclitaxel SST-fusion protein conjugated complex were able to inhibit SST and SST2 receptor binding with IC₅₀ value of 6.55 nM.

TABLE 6
Inhibition of SST binding onto SST2A receptor by paclitaxel SST-fusion
protein (SEQ ID NO: 1) conjugated complex

| Compound | IC₅₀ | Kᵢ | n_{H} |
|---|---|---|---|
| Paclitaxel-SST fusion protein Conjugated | 6.55 nM | 3.48 nM | 2.61 |
| Complex | | | |
| SST 14 | 5.43 pM | 2.88 pM | 0.79 |

### Example 17: Paclitaxel SST-Fusion Protein (SEQ ID NO: 1) Conjugated Complex Binding onto SST2A Receptor in Cho-K1 Cells

CHO-K1 cells expressing sst2a receptor were used in an adenylyl cyclase assay to quantitatively determine the binding of paclitaxel SST-fusion protein conjugated complex to sst2a receptor. Various concentration of paclitaxel SST-fusion protein (SEQ ID NO: 1) were tested at 1 nM-0.3 μM. Paclitaxel SST-fusion protein conjugated complex was able to bind to SST2a receptor expressed CHO-K1 cells with EC₅₀ value of 8.29 nM (Table 7).

TABLE 7
Paclitaxel SST-fusion protein (SEQ ID NO: 1) conjugated complex
binding onto SST2A receptor in CHO-K1 cells

| Compound | EC₅₀ |
|---|---|
| Paclitaxel-SST fusion protein Conjugated Complex | 8.29 nM |
| Octreotide | 0.039 nM |

1. Particles comprising a pharmacologically active ingredient, or a diagnostic ingredient, and a polymeric shell, wherein the polymeric shell comprises a somatostatin-albumin fusion protein, and the polymeric shell encapsulates the pharmacologically active ingredient, or the diagnostic ingredient.

2. The particles according to claim 1, wherein the polymeric shell comprises from about 5 percent to about 100 percent or from about 65 percent to about 95 percent, by weight, of a somatostatin-albumin fusion protein.

3. (canceled)

4. The particles according to claim 1, wherein the pharmacologically active ingredient is an anticancer agent, a nutritional agent, or a nutraceutical.

5. The particles according to claim 4, wherein the anticancer agent is selected from the group consisting of nitrogen mustard, nitrosourea, ethyleneimine, alkane sulfonates, tetrazine, platinum compounds, pyrimidine analogs, purine analogs, antimetabolites, folate analogs, anthracyclines, taxane, vinca alkaloid, topoisomerase inhibitor, hormonal agent, and combinations thereof.

6. (canceled)

7. The particles according to claim 1, wherein the somatostatin-albumin fusion protein comprises:
an SST;
an L; and
an ALB, that are operably connected,
wherein,
L connects SST and ALB, in any order,
SST is a somatostatin, its analogue or derivative;
L is a spacer or a linker; and
ALB is an albumin, its analogue or variant,
wherein L connects SST and ALB, in any order.

8. The particles according to claim 7, wherein the fusion protein is selected from the group consisting of:
SST-(L)_{X1}-ALB (I);
ALB-(L)ₓ₁-SST (II);
[SST-(L)ₓ₁]_{y1}-ALB (III);
ALB-[(L)ₓ₁-SST]_{y1} (IV);
[SST-(L)ₓ₁]_{y1}-ALB-[(L)ₓ₂-SST]_{y2} (V);
[SST-(L)ₓ₁]_{y1}-ALB-[(L)ₓ₂-SST]_{y2}-(L)ₓ₃-ALB (VI);
[SST-(L)ₓ₁]_{y1}-ALB-[(L)ₓ₂-SST]_{y2}-(L)ₓ₃-ALB-[(L)ₓ₄-S ST]_{y3} (VII);
ALB-(L)ₓ₁-[SST-(L)ₓ₂]_{y1}-ALB (VIII);
ALB-(L)ₓ₁-[SST-(L)ₓ₂]_{y1}-ALB-[(L)ₓ₃-SST]_{y2}-(L)ₓ₁-ALB (IX); and
ALB-(L)ₓ₁-[S ST-(L)ₓ₂]_{y1}-ALB-[(L)ₓ₃-SST]_{y2}-(L)ₓ₁-ALB-[(L)ₓ₄-SST]_{y3} (X);
wherein, x1, x2, x3, x4, y1, y2, or y3 is independently zero or an integer selected from 1-10.

9. The particles according to claim 7, wherein the SST is either naturally occurring or synthetically manufactured.

10. The particles according to claim 7, wherein the SST comprises one or more tandem repeats of a sequence encoding SST-14 or SST-28, represented by SEQ ID NOS: 17 or 18, respectively, or a sequence having at least 85% identity to either of these sequences.

11. (canceled)

12. The particles according to claim 7, wherein the L is either a flexible or an alpha helically structured polypeptide linker or spacer.

13. (canceled)

14. (canceled)

15. (canceled)

16. (canceled)

17. (canceled)

18. (canceled)

19. The particles according to claim 1, wherein the somatostatin-albumin fusion protein is substantially crosslinked by way of disulfide bonds.

20. (canceled)

21. The particles according to claim 1, wherein the polymeric shell substantially contains the pharmacologically active agent.

22. The particles according to claim 21, wherein the largest cross-sectional dimension of said polymeric shell is from about 0.001 micron to about 1000 micron or from about 0.01 micron to about 1.0 micron.

23. (canceled)

24. (canceled)

25. (canceled)

26. (canceled)

27. (canceled)

28. The particles according to claim 4, wherein nutritional agents is selected from the group consisting of amino acids, sugars, proteins, carbohydrates, fat-soluble vitamins, fat, oil and combinations thereof.

29. (canceled)

30. (canceled)

31. A method for the delivery of substantially water insoluble pharmaceutical agents to a subject, said method comprising administering to said subject an effective amount of the particles of claim 1.

32. A method for preparing particles comprising pharmaceutically active ingredients, comprising: subjecting an aqueous medium containing a somatostatin-albumin fusion protein and a pharmaceutically active agent to shear conditions for a time sufficient to promote crosslinking of the somatostatin-albumin fusion protein by disulfide bonds to produce a polymeric shell containing the pharmacologically active agent therein.

33. The method according to claim 32, wherein the pharmaceutically active agent is an anticancer agent that is selected from the group consisting of nitrogen mustard, nitrosoruea, ethyleneimine, alkane sulfonates, tetrazine, platinum compounds, pyrimidine analogs, purine analogs, antimetabolites, folate analogs, anthracyclines, taxane, vinca alkaloid, topoisomerase inhibitor, hormonal agent, and combinations thereof.

34. (canceled)

35. (canceled)

36. (canceled)

37. (canceled)

38. (canceled)

39. (canceled)

40. The particles of claim 1, wherein the weight ratio of the SST fusion protein and the pharmacologically active ingredient, or the diagnostic ingredient, in the particles is about 20:1 to 1:20.

41. (canceled)

42. (canceled)

43. A pharmaceutical composition comprising the particles of claim 1, and a physiologically acceptable excipient or carrier.

44. A method of treating or diagnosing a cancer comprising administering an effective amount of the particles of claim 1 to a subject in need thereof.

45. A method of treating or diagnosing a cancer comprising administering an effective amount of the pharmaceutical composition of claim 43 to a subject in need thereof.


SEQUENCE LISTING (46 sequences)

SEQ ID NO: 1 - protein, 657 aa, Artificial Sequence
  SST14-A(EAAAK)4A-HSA-A(EAAAK)4A-SST14
AGCKNFFWKTFTSCAEAAAKEAAAKEAAAKEAAAKADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKEAAAKEAAAKEAAAKAAGCKNFFWKTFTSC

SEQ ID NO: 2 - protein, 621 aa, Artificial Sequence
  HSA-A(EAAAK)4A-SST14
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKEAAAKEAAAKEAAAKAAGCKNFFWKTFTSC

SEQ ID NO: 3 - protein, 1205 aa, Artificial Sequence
  His6-GGGGS-HSA-GGGGS-SST14-HSA
HHHHHHGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSAGCKNFFWKTFTSCGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 4 - protein, 1224 aa, Artificial Sequence
  His6-GGGGS-HSA-GGGGS-(SST14-GGGGS)2-HSA
HHHHHHGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSAGCKNFFWKTFTSCGGGGSAGCKNFFWKTFTSCGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 5 - protein, 1213 aa, Artificial Sequence
  HSA-GGGGS-(SST14-GGGGS)2- HSA
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSAGCKNFFWKTFTSCGGGGSAGCKNFFWKTFTSCGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 6 - protein, 8 aa, Artificial Sequence
  Linker
GGGGGGGG

SEQ ID NO: 7 - protein, 614 aa, Artificial Sequence
  SST14-(GGGGS)3-HSA
AGCKNFFWKTFTSCGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 8 - protein, 616 aa, Artificial Sequence
  SST14-A(EAAAK)4A-HSA
AGCKNFFWKTFTSCAEAAAKEAAAKEAAAKADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 9 - protein, 615 aa, Artificial Sequence
  His6-GGGGS-HSA-GGGGS-SST14
HHHHHHGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSAGCKNFFWKTFTSC

SEQ ID NO: 10 - protein, 615 aa, Artificial Sequence
  SST14-GGGGS-HSA-GGGGS-His6
AGCKNFFWKTFTSCGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSHHHHHH

SEQ ID NO: 11 - protein, 604 aa, Artificial Sequence
  HSA-GGGGS-SST14
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSAGCKNFFWKTFTSC

SEQ ID NO: 12 - protein, 604 aa, Artificial Sequence
  SST14-GGGGS-HSA
AGCKNFFWKTFTSCGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 13 - protein, 623 aa, Artificial Sequence
  (SST14-GGGGS)2- HSA
AGCKNFFWKTFTSCGGGGSAGCKNFFWKTFTSCGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 14 - protein, 661 aa, Artificial Sequence
  (SST14-GGGGS)4- HSA
AGCKNFFWKTFTSCGGGGSAGCKNFFWKTFTSCGGGGSAGCKNFFWKTFTSCGGGGSAGCKNFFWKTFTSCGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 15 - protein, 614 aa, Artificial Sequence
  HSA-(GGGGS)3-SST14
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAGCKNFFWKTFTSC

SEQ ID NO: 16 - protein, 629 aa, Artificial Sequence
  HSA-(GGGGS)6-SST14
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSGGGGSGGGGSGGGGSAGCKNFFWKTFTSC

SEQ ID NO: 17 - protein, 14 aa, Homo sapiens
AGCKNFFWKTFTSC

SEQ ID NO: 18 - protein, 28 aa, Homo sapiens
SANSNPAMAPRERKAGCKNFFWKTFTSC

SEQ ID NO: 19 - protein, 585 aa, Homo sapiens [sequence omitted]

SEQ ID NO: 20 - protein, 22 aa, Homo sapiens
MDMRVPAQLLGLLLLWLRGARC

SEQ ID NO: 21 - protein, 20 aa, Artificial Sequence
  Linker
APAPAPAPAPAPAPAPAPAP

SEQ ID NO: 22 - protein, 40 aa, Artificial Sequence
  Linker
APAPAPAPAPAPAPAPAPAPAPAPAPAPAPAPAPAPAPAP

SEQ ID NO: 23 - DNA, 42 bp, Homo sapiens
gctggctgcaagaatttcttctggaagactttcacatcctgt

SEQ ID NO: 24 - DNA, 70 bp, Homo sapiens
tctgctaactcaaacccggctatggcaccccgagaacgcaaagctggctgcaagaatttcttctggaaga

SEQ ID NO: 25 - DNA, 1755 bp, Homo sapiens
gatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggctta

SEQ ID NO: 26 - DNA, 5428 bp, Artificial Sequence
  pcDNA3.1(+) Vector
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 27 - DNA, 9276 bp, Artificial Sequence
  pPIC9K Vector
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatccaaacgatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagcttacgtagaattccctagggcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaagttcgtttgtgcaagcttatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctatcgaatctaaatgtaagttaaaatctctaaataattaaataagtcccagtttctccatacgaaccttaacagcattgcggtgagcatctagaccttcaacagcagccagatccatcactgcttggccaatatgtttcagtccctcaggagttacgtcttgtgaagtgatgaacttctggaaggttgcagtgttaactccgctgtattgacgggcatatccgtacgttggcaaagtgtggttggtaccggaggagtaatctccacaactctctggagagtaggcaccaacaaacacagatccagcgtgttgtacttgatcaacataagaagaagcattctcgatttgcaggatcaagtgttcaggagcgtactgattggacatttccaaagcctgctcgtaggttgcaaccgatagggttgtagagtgtgcaatacacttgcgtacaatttcaacccttggcaactgcacagcttggttgtgaacagcatcttcaattctggcaagctccttgtctgtcatatcgacagccaacagaatcacctgggaatcaataccatgttcagcttgagacagaaggtctgaggcaacgaaatctggatcagcgtatttatcagcaataactagaacttcagaaggcccagcaggcatgtcaatactacacagggctgatgtgtcattttgaaccatcatcttggcagcagtaacgaactggtttcctggaccaaatattttgtcacacttaggaacagtttctgttccgtaagccatagcagctactgcctgggcgcctcctgctagcacgatacacttagcaccaaccttgtgggcaacgtagatgacttctggggtaagggtaccatccttcttaggtggagatgcaaaaacaatttctttgcaaccagcaactttggcaggaacacccagcatcagggaagtggaaggcagaattgcggttccaccaggaatatagaggccaactttctcaataggtcttgcaaaacgagagcagactacaccagggcaagtctcaacttgcaacgtctccgttagttgagcttcatggaatttcctgacgttatctatagagagatcaatggctctcttaacgttatctggcaattgcataagttcctctgggaaaggagcttctaacacaggtgtcttcaaagcgactccatcaaacttggcagttagttctaaaagggctttgtcaccattttgacgaacattgtcgacaattggtttgactaattccataatctgttccgttttctggataggacgacgaagggcatcttcaatttcttgtgaggaggccttagaaacgtcaattttgcacaattcaatacgaccttcagaagggacttctttaggtttggattcttctttaggttgttccttggtgtatcctggcttggcatctcctttccttctagtgacctttagggacttcatatccaggtttctctccacctcgtccaacgtcacaccgtacttggcacatctaactaatgcaaaataaaataagtcagcacattcccaggctatatcttccttggatttagcttctgcaagttcatcagcttcctccctaattttagcgttcaacaaaacttcgtcgtcaaataaccgtttggtataagaaccttctggagcattgctcttacgatcccacaaggtggcttccatggctctaagaccctttgattggccaaaacaggaagtgcgttccaagtgacagaaaccaacacctgtttgttcaaccacaaatttcaagcagtctccatcacaatccaattcgatacccagcaacttttgagttgctccagatgtagcacctttataccacaaaccgtgacgacgagattggtagactccagtttgtgtccttatagcctccggaatagactttttggacgagtacaccaggcccaacgagtaattagaagagtcagccaccaaagtagtgaatagaccatcggggcggtcagtagtcaaagacgccaacaaaatttcactgacagggaactttttgacatcttcagaaagttcgtattcagtagtcaattgccgagcatcaataatggggattataccagaagcaacagtggaagtcacatctaccaactttgcggtctcagaaaaagcataaacagttctactaccgccattagtgaaacttttcaaatcgcccagtggagaagaaaaaggcacagcgatactagcattagcgggcaaggatgcaactttatcaaccagggtcctatagataaccctagcgcctgggatcatcctttggacaactctttctgccaaatctaggtccaaaatcacttcattgataccattattgtacaacttgagcaagttgtcgatcagctcctcaaattggtcctctgtaacggatgactcaacttgcacattaacttgaagctcagtcgattgagtgaacttgatcaggttgtgcagctggtcagcagcatagggaaacacggcttttcctaccaaactcaaggaattatcaaactctgcaacacttgcgtatgcaggtagcaagggaaatgtcatacttgaagtcggacagtgagtgtagtcttgagaaattctgaagccgtatttttattatcagtgagtcagtcatcaggagatcctctacgccggacgcatcgtggccgacctgcagggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccaaggcgataaatacccaaatctaaaactcttttaaaacgttaaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattctcatgtttgacagcttatcatcgataagctgactcatgttggtattgtgaaatagacgcagatcgggaacactgaaaaataacagttattattcg

SEQ ID NO: 28 - protein, 22 aa, Artificial Sequence
  Linker
AEAAAKEAAAKEAAAKEAAAKA

SEQ ID NO: 29 - protein, 5 aa, Artificial Sequence
  Linker
GGGGS

SEQ ID NO: 30 - protein, 16 aa, Artificial Sequence
  Linker
GGGGSLVPRGSGGGGS

SEQ ID NO: 31 - protein, 6 aa, Artificial Sequence
  Linker
GSGSGS

SEQ ID NO: 32 - protein, 15 aa, Artificial Sequence
  Linker
GGGGSLVPRGSGGGG

SEQ ID NO: 33 - protein, 16 aa, Artificial Sequence
  Linker
GGGGSLVPRGSGGGGS

SEQ ID NO: 34 - protein, 11 aa, Artificial Sequence
  Linker
GGSGGHMGSGG

SEQ ID NO: 35 - protein, 11 aa, Artificial Sequence
  Linker
GGSGGSGGSGG

SEQ ID NO: 36 - protein, 11 aa, Artificial Sequence
  Linker
GGSGGHMGSGG

SEQ ID NO: 37 - protein, 5 aa, Artificial Sequence
  Linker
GGSGG

SEQ ID NO: 38 - protein, 16 aa, Artificial Sequence
  Linker
GGGGSLVPRGSGGGGS

SEQ ID NO: 39 - protein, 8 aa, Artificial Sequence
  Linker
GGSGGGGG

SEQ ID NO: 40 - protein, 8 aa, Artificial Sequence
  Linker
GSGSGSGS

SEQ ID NO: 41 - protein, 18 aa, Artificial Sequence
  Linker
GGGSEGGGSEGGGSEGGG

SEQ ID NO: 42 - protein, 8 aa, Artificial Sequence
  Linker
AAGAATAA

SEQ ID NO: 43 - protein, 5 aa, Artificial Sequence
  Linker
GGGGG

SEQ ID NO: 44 - protein, 5 aa, Artificial Sequence
  Linker
GGSSG

SEQ ID NO: 45 - protein, 11 aa, Artificial Sequence
  Linker
GSGGGTGGGSG

SEQ ID NO: 46 - protein, 26 aa, Artificial Sequence
  Linker
GSGSGSGSGGSGGSGGSGGSGGSGGS